(19) United States
(12) Patent Application Publication
(10) Pub. No.: US 2017/0281758 A1
(43) Pub. Date: Oct. 5, 2017

(54) CALICHEAMICIN ANTIBODY DRUG CONJUGATES LINKING AN AMIDOACETYL GROUP TO A SUGAR MOIETY ON CALICHEAMICIN

(71) Applicant: Sorrento Therapeutics, Inc., San Diego, CA (US)

(72) Inventors: Gang Chen, San Diego, CA (US); Alisher B. Khasanov, San Diego, CA (US); Hong Zhang, San Diego, CA (US); Tong Zhu, San Diego, CA (US); Zhenwei Miao, San Diego, CA (US)

(21) Appl. No.: 15/472,965

(22) Filed: Mar. 29, 2017

Related U.S. Application Data
(60) Provisional application No. 62/314,799, filed on Mar. 29, 2016.

(51) Int. Cl. A61K 39/395; A61K 31/44; A61K 31/4192; A61K 31/4985

(52) U.S. Cl. CPC A61K 39/395; A61K 31/4985; A61K 31/44; A61K 31/4192; C07K 9/00

(57) ABSTRACT

There is disclosed a calicheamicin antibody drug conjugate comprising a linking amidoacetyl group covalently bound to a sugar moiety on calicheamicin or linking to sulfur atom on calicheamicin through disulfide bond.

### RELATED APPLICATIONS

This application claims priority to U.S. Provisional Application No. 62/314,799, filed Mar. 29, 2016, the entire contents of which are incorporated herein by reference.

### TECHNICAL FIELD

The present disclosure provides a calicheamicin antibody drug conjugate comprising a linking amidoacetyl group covalently bound to a sugar moiety on calicheamicin or linking to sulfur atom on calicheamicin through disulfide bond.

### BACKGROUND

Calicheamicin is described in WO 03/092623 as an ADC (antibody drug conjugate). The synthesis of calicheamicin was published in 2002 (Bioconjugate Chem. 2002, 13, 47-58). ADCs (antibody drug conjugates) that used calicheamicin as the drug moiety linked an acid sensitive hydrazone linker attached to N-acetyl gamma calicheamicin via a disulfide bond to attach calicheamicin to the rest of the conjugate. As shown in formula I below of N-acetyl gamma calicheamicin linked an antibody that is shown as “Ab.”

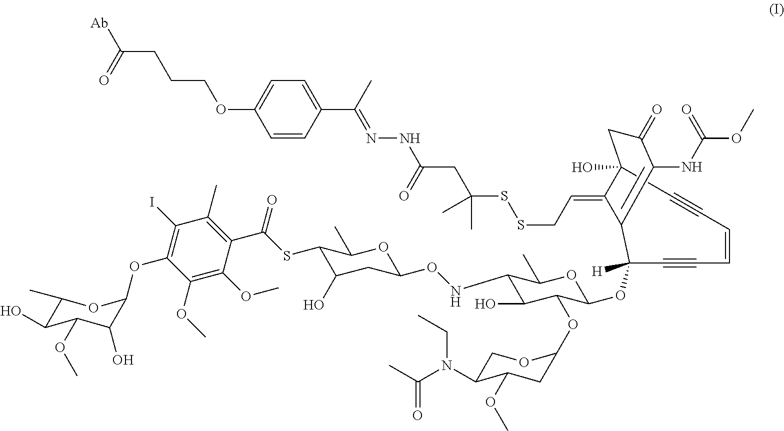

However, because of the instability of this hydrazone linker, the calicheamicin toxin was released from the linker at a high rate of 6% in 24 h at 37° C. This high rate of release caused large amounts of non-specific cytotoxicity with such calicheamicin ADCs. Therefore, there is a need in the art to find a better way to link calicheamicin to a linker and a targeting antibody such that the calicheamicin will be released from the linker at a much slower rate to lower non-specific cytotoxicity. The present disclosure provides a solution to utilizing calicheamicin in an ADC construct while significantly lowering the rate of calicheamicin release from the linker to significantly improve non-specific cytotoxity side effects.

### SUMMARY

The present disclosure provides a calicheamicin antibody drug conjugate (ADC) comprising a linking amidoacetyl group covalently bound to a sugar moiety on calicheamicin or linking to sulfur atom on calicheamicin through disulfide bond. More specifically, the present disclosure provides an ADC comprising a structure of Formula II

AbL¹-L²-D)ₙ (II)
or a pharmaceutically acceptable salt thereof, wherein:

Ab is a monoclonal antibody;

L¹-L² together are a linker selected from the group consisting of:

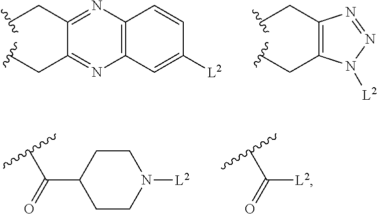

wherein the wavy line indicates a point of attachment to an Ab;

L² is a linker; wherein L² is selected from the group consisting of an amino acid, a peptide, —(CH₂)ₘ—, —(CH₂CH₂O)ₘ—, PAB (p-aminobenzyl), Val (Valine)-Citrulline-PAB, Val-Ala (Alanine)-PAB, Ala-Ala-Asn-PAB, and combinations thereof, wherein m is an integer from 0 to 10;

D is calicheamicin; and
n is 1, 2, 3, 4, 5, 6, 7, 8, 9, or 10.

Preferably, D has the structure of Formula III

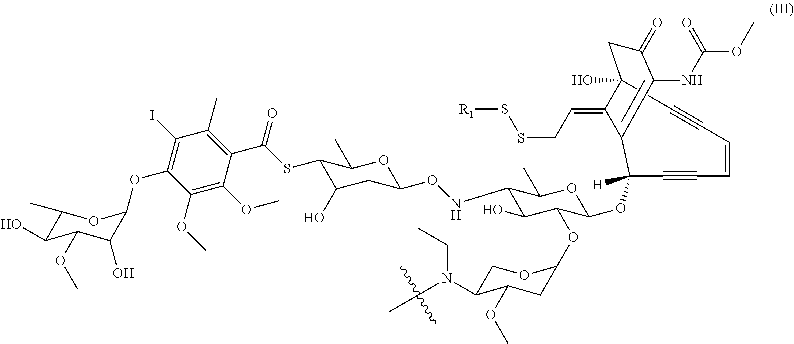

wherein the wavy line indicates the point of attachment to L²;
wherein R1 is selected from the group consisting of C1-C8 alkyl, —(CH₂CH₂O)ₙ—, isopropyl, glucose, galactose, mannose, glucosamine, C1-C8 alkyl-OH, and combinations thereof.

More preferably the D and C components of the ADC is the structure of Formula IV:

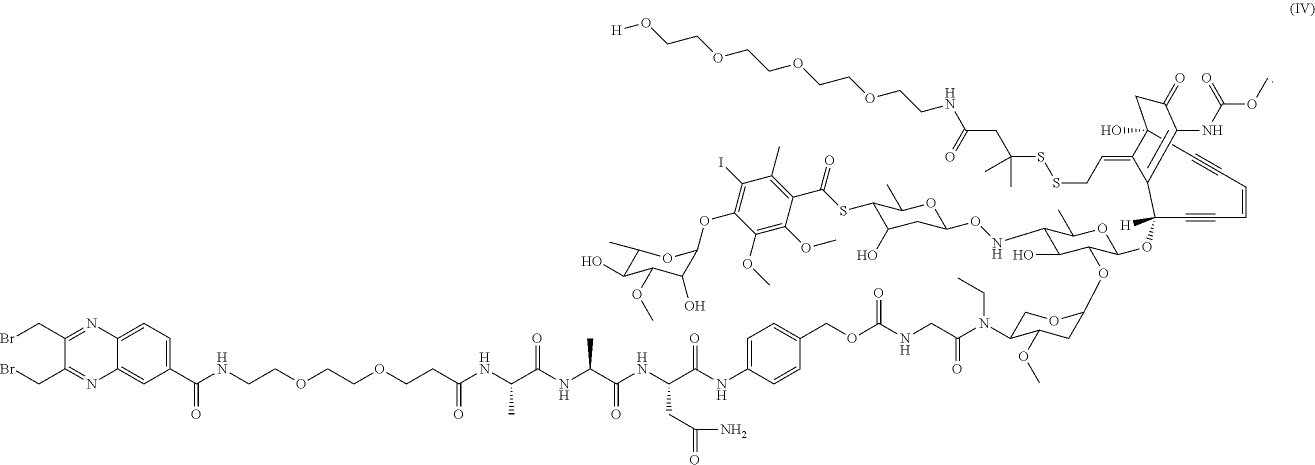 [SMILES: CCN(C(=O)CNC(=O)OCc1ccc(NC(=O)[C@H](CC(N)=O)NC(=O)[C@H](C)NC(=O)[C@H](C)NC(=O)CCOCCOCCNC(=O)c2ccc3nc(CBr)c(CBr)nc3c2)cc1)C1COC(OC2C(O[C@H]3C#C/C=C\C#C[C@]4(O)CC(=O)C(NC(=O)OC)=C3/C4=C\CSSC(C)(C)CC(=O)NCCOCCOCCOCCO)OC(C)C(NOC3CC(O)C(SC(=O)c4c(C)c(I)c(OC5OC(C)C(O)C(OC)C5O)c(OC)c4OC)C(C)O3)C2O)CC1OC]

The linker was attached via a stable amide bond or a carbamate bond. A hydrophilic group (e.g., PEG4) was incorporated via a disulfide bond to improve the solubility in aqueous buffer and mitigate the issue of aggregation.

### BRIEF DESCRIPTION OF THE FIGURES

FIG. 1 illustrates the tumor volume from 3 mg/kg IV, single dose of Compound 81a in HCC827 tumor in nude mice.

FIG. 2 illustrates the tumor volume from 3 mg/kg IV, single dose of Compound 81a in H292 tumor in nude mice.

FIG. 3 illustrates the tumor volume from 3 mg/kg IV, one weekly for three treatments of Compound 81a in H292 tumor in nude mice.

FIG. 4 illustrates the tumor volume from 3 mg/kg IV, one weekly for three treatments of Compound 81a in H1993 tumor in nude mice.

FIG. 5 illustrates the tumor volume from 3 mg/kg IV, one weekly for three treatments of Compound81a in U87MG tumor in nude mice.

FIG. 6 illustrates the tumor volume from 3 mg/kg IV, single dose of Compound 8a in HCC827 tumor in nude mice.

FIG. 7 illustrates the tumor volume from 3 mg/kg IV, single dose of Compound 8a in H292 tumor in nude mice.

FIG. 8 illustrates the tumor volume from 3 mg/kg IV, single dose of Compound 8a in U87 tumor in nude mice.

FIG. 9 illustrates the tumor volume from 3 mg/kg IV, single dose of Compound 8a in H1975 tumor in nude mice.

### DETAILED DESCRIPTION

Examples of Formula III, where R1 and L¹-L² are listed below (wavy line indicates point of attachment to Formula III):

| R1 | L¹-L² |
|---|---|
| isopropyl | 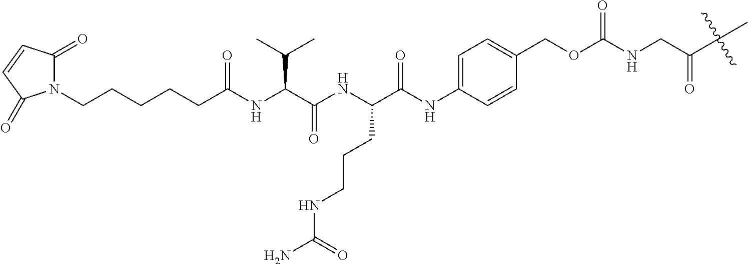 [SMILES: *C(=O)CNC(=O)OCc1ccc(NC(=O)[C@H](CCCNC(N)=O)NC(=O)[C@@H](NC(=O)CCCCCN2C(=O)C=CC2=O)C(C)C)cc1] |
| isopropyl | 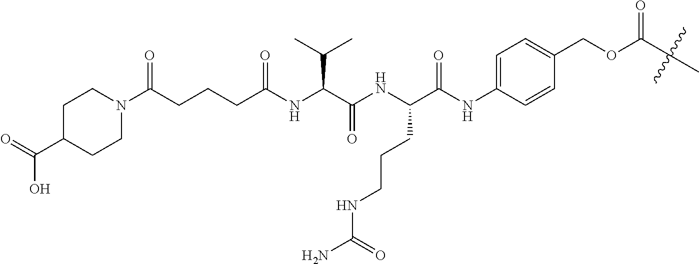 [SMILES: *C(=O)OCc1ccc(NC(=O)[C@H](CCCNC(N)=O)NC(=O)[C@@H](NC(=O)CCCC(=O)N2CCC(C(=O)O)CC2)C(C)C)cc1] |
| isopropyl | 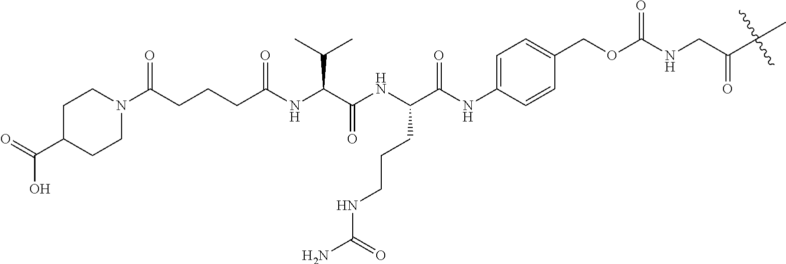 [SMILES: *C(=O)CNC(=O)OCc1ccc(NC(=O)[C@H](CCCNC(N)=O)NC(=O)[C@@H](NC(=O)CCCC(=O)N2CCC(C(=O)O)CC2)C(C)C)cc1] |
| isopropyl | 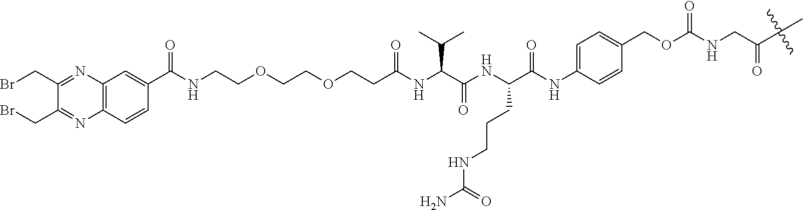 [SMILES: *C(=O)CNC(=O)OCc1ccc(NC(=O)[C@H](CCCNC(N)=O)NC(=O)[C@@H](NC(=O)CCOCCOCCNC(=O)c2ccc3nc(CBr)c(CBr)nc3c2)C(C)C)cc1] |
| isopropyl | 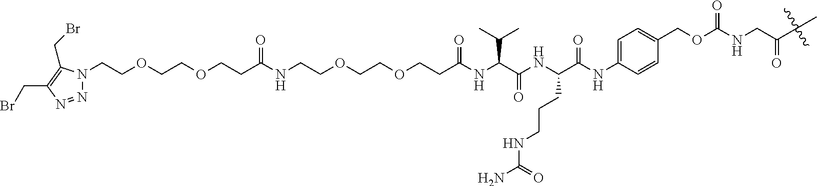 [SMILES: *C(=O)CNC(=O)OCc1ccc(NC(=O)[C@H](CCCNC(N)=O)NC(=O)[C@@H](NC(=O)CCOCCOCCNC(=O)CCOCCOCCn2nnc(CBr)c2CBr)C(C)C)cc1] |
| 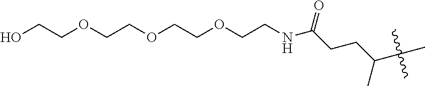 [SMILES: *C(C)CCC(=O)NCCOCCOCCOCCO] | 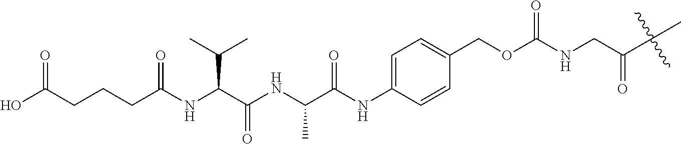 [SMILES: *C(=O)CNC(=O)OCc1ccc(NC(=O)[C@H](C)NC(=O)[C@@H](NC(=O)CCCC(=O)O)C(C)C)cc1] |
| 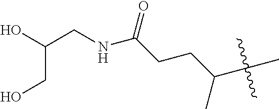 [SMILES: *C(C)CCC(=O)NCC(O)CO] | 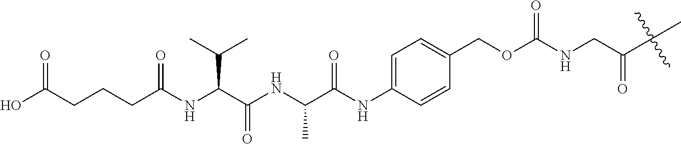 [SMILES: *C(=O)CNC(=O)OCc1ccc(NC(=O)[C@H](C)NC(=O)[C@@H](NC(=O)CCCC(=O)O)C(C)C)cc1] |
| 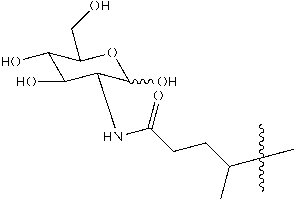 [SMILES: *C(C)CCC(=O)NC1C(O)OC(CO)C(O)C1O] | 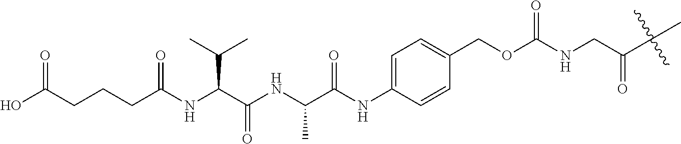 [SMILES: *C(=O)CNC(=O)OCc1ccc(NC(=O)[C@H](C)NC(=O)[C@@H](NC(=O)CCCC(=O)O)C(C)C)cc1] |
| tert-butyl | 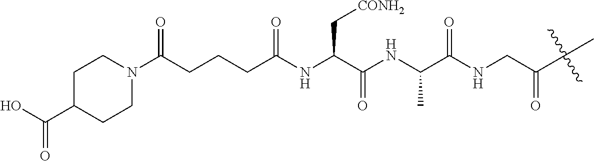 [SMILES: *C(=O)CNC(=O)[C@H](C)NC(=O)[C@H](CC(N)=O)NC(=O)CCCC(=O)N1CCC(C(=O)O)CC1] |
| isopropyl | 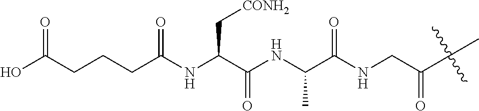 [SMILES: *C(=O)CNC(=O)[C@H](C)NC(=O)[C@H](CC(N)=O)NC(=O)CCCC(=O)O] |
| tert-butyl | 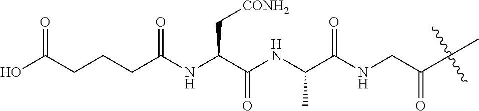 [SMILES: *C(=O)CNC(=O)[C@H](C)NC(=O)[C@H](CC(N)=O)NC(=O)CCCC(=O)O] |
| 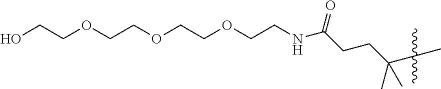 [SMILES: *C(C)(C)CCC(=O)NCCOCCOCCOCCO] | 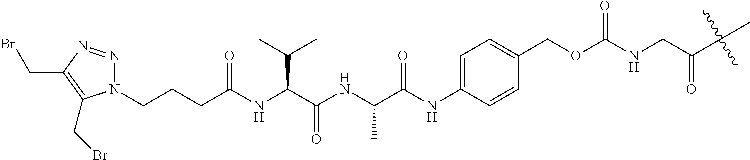 [SMILES: *C(=O)CNC(=O)OCc1ccc(NC(=O)[C@H](C)NC(=O)[C@@H](NC(=O)CCCn2nnc(CBr)c2CBr)C(C)C)cc1] |
| 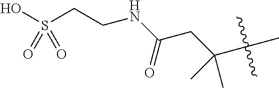 [SMILES: *C(C)(C)CC(=O)NCCS(=O)(=O)O] | 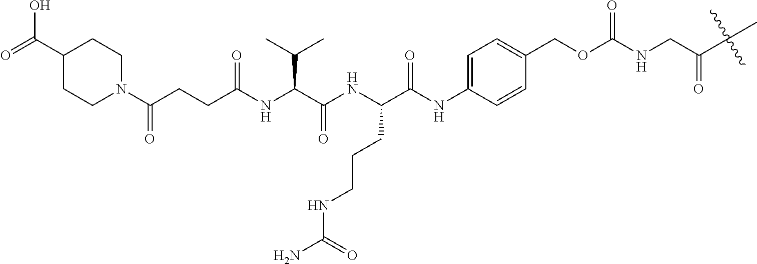 [SMILES: *C(=O)CNC(=O)OCc1ccc(NC(=O)[C@H](CCCNC(N)=O)NC(=O)[C@@H](NC(=O)CCC(=O)N2CCC(C(=O)O)CC2)C(C)C)cc1] |
| 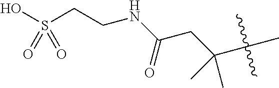 [SMILES: *C(C)(C)CC(=O)NCCS(=O)(=O)O] | 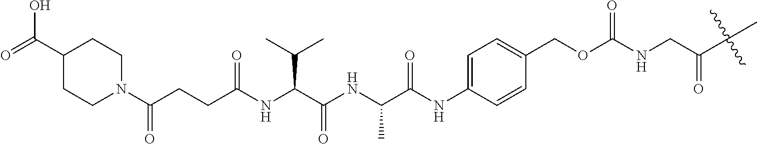 [SMILES: *C(=O)CNC(=O)OCc1ccc(NC(=O)[C@H](C)NC(=O)[C@@H](NC(=O)CCC(=O)N2CCC(C(=O)O)CC2)C(C)C)cc1] |
| 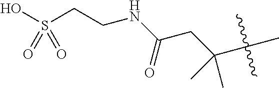 [SMILES: *C(C)(C)CC(=O)NCCS(=O)(=O)O] | 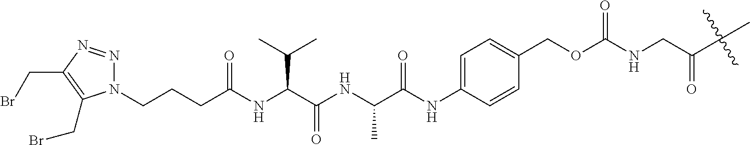 [SMILES: *C(=O)CNC(=O)OCc1ccc(NC(=O)[C@H](C)NC(=O)[C@@H](NC(=O)CCCn2nnc(CBr)c2CBr)C(C)C)cc1] |
| 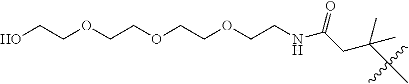 [SMILES: *C(C)(C)CC(=O)NCCOCCOCCOCCO] | 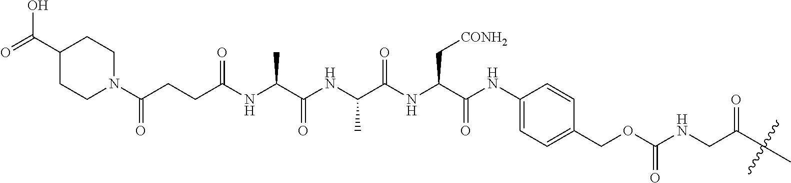 [SMILES: *C(=O)CNC(=O)OCc1ccc(NC(=O)[C@H](CC(N)=O)NC(=O)[C@H](C)NC(=O)[C@H](C)NC(=O)CCC(=O)N2CCC(C(=O)O)CC2)cc1] |
| 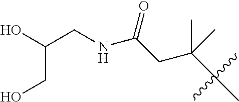 [SMILES: *C(C)(C)CC(=O)NCC(O)CO] | 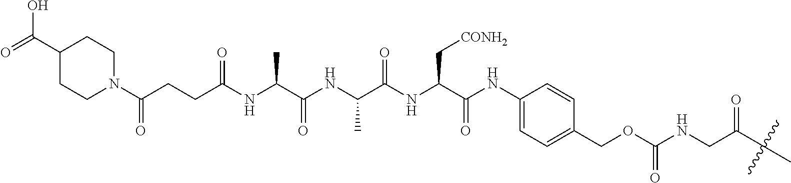 [SMILES: *C(=O)CNC(=O)OCc1ccc(NC(=O)[C@H](CC(N)=O)NC(=O)[C@H](C)NC(=O)[C@H](C)NC(=O)CCC(=O)N2CCC(C(=O)O)CC2)cc1] |
| tert-butyl | 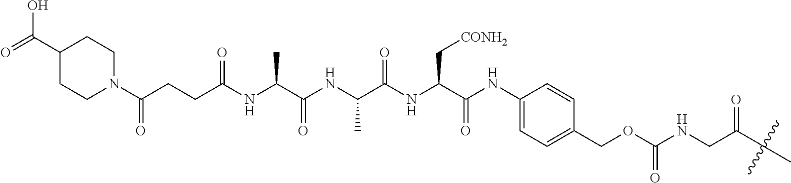 [SMILES: *C(=O)CNC(=O)OCc1ccc(NC(=O)[C@H](CC(N)=O)NC(=O)[C@H](C)NC(=O)[C@H](C)NC(=O)CCC(=O)N2CCC(C(=O)O)CC2)cc1] |
| 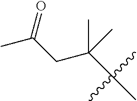 [SMILES: *C(C)(C)CC(C)=O] | 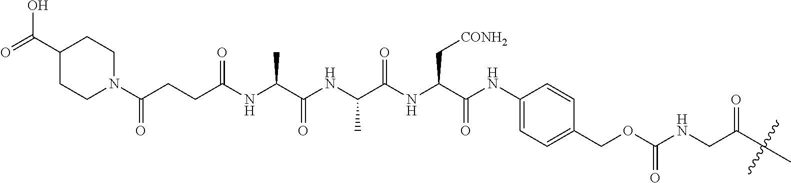 [SMILES: *C(=O)CNC(=O)OCc1ccc(NC(=O)[C@H](CC(N)=O)NC(=O)[C@H](C)NC(=O)[C@H](C)NC(=O)CCC(=O)N2CCC(C(=O)O)CC2)cc1] |
| 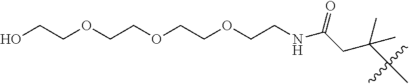 [SMILES: *C(C)(C)CC(=O)NCCOCCOCCOCCO] | 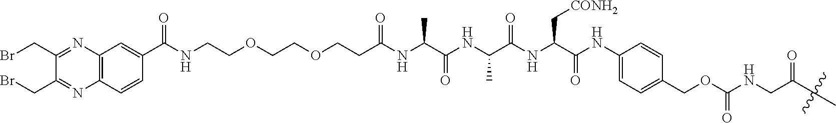 [SMILES: *C(=O)CNC(=O)OCc1ccc(NC(=O)[C@H](CC(N)=O)NC(=O)[C@H](C)NC(=O)[C@H](C)NC(=O)CCOCCOCCNC(=O)c2ccc3nc(CBr)c(CBr)nc3c2)cc1] |
| 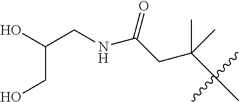 [SMILES: *C(C)(C)CC(=O)NCC(O)CO] | 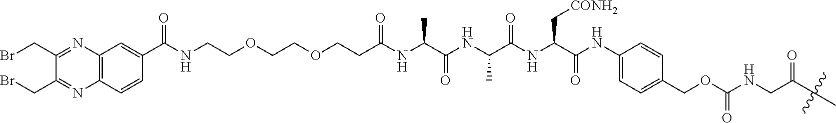 [SMILES: *C(=O)CNC(=O)OCc1ccc(NC(=O)[C@H](CC(N)=O)NC(=O)[C@H](C)NC(=O)[C@H](C)NC(=O)CCOCCOCCNC(=O)c2ccc3nc(CBr)c(CBr)nc3c2)cc1] |
| tert-butyl | 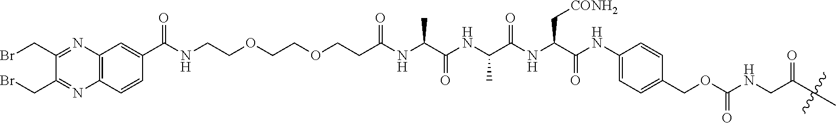 [SMILES: *C(=O)CNC(=O)OCc1ccc(NC(=O)[C@H](CC(N)=O)NC(=O)[C@H](C)NC(=O)[C@H](C)NC(=O)CCOCCOCCNC(=O)c2ccc3nc(CBr)c(CBr)nc3c2)cc1] |
| 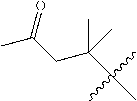 [SMILES: *C(C)(C)CC(C)=O] | 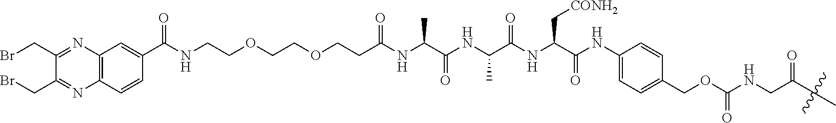 [SMILES: *C(=O)CNC(=O)OCc1ccc(NC(=O)[C@H](CC(N)=O)NC(=O)[C@H](C)NC(=O)[C@H](C)NC(=O)CCOCCOCCNC(=O)c2ccc3nc(CBr)c(CBr)nc3c2)cc1] |

### DEFINITIONS

As used herein, common organic abbreviations are defined as follows:
Ac Acetyl
ACN Acetonitrile
Ala Alanine
Asn Asparagine
aq. Aqueous
BOC or Boc tert-Butoxycarbonyl
° C. Temperature in degrees Centigrade
Cit Citrulline
DCM dichloromethane
DIEA Diisopropylethylamine
DMF N, N′-Dimethylformamide
EDC 1-Ethyl-3-(3-dimethylaminopropyl)carbodiimide
Et Ethyl
EtOAc Ethyl acetate
Eq Equivalents
Fmoc 9-Fluorenylmethoxycarbonyl
g Gram(s)
h Hour (hours)
HATU 2-(1H-7-azabenzotriazol-1-yl)-1,1,3,3-tetramethyl uronium hexafluorophosphate
HOBt N-Hydroxybenzotriazole
HPLC High-performance liquid chromatography
LC/MS Liquid chromatography-mass spectrometry
Me Methyl
mg milligrams
MeOH Methanol
mL Milliliter(s)
μL/μL Microliter(s)
mol moles
mmol millimoles
μmol/umol micromoles
MS mass spectrometry
NHS N-Hydroxysuccinimide
PAB p-aminobenzyl
Pip piperidine
RP-HPLC reverse phase HPLC
rt room temperature
t-Bu tert-Butyl
Tert, t tertiary
TFA Trifluoracetic acid
THF Tetrahydrofuran
Val Valine

### SYNTHESIS EXAMPLE 1

### Preparation of Compound 8 and 8a:

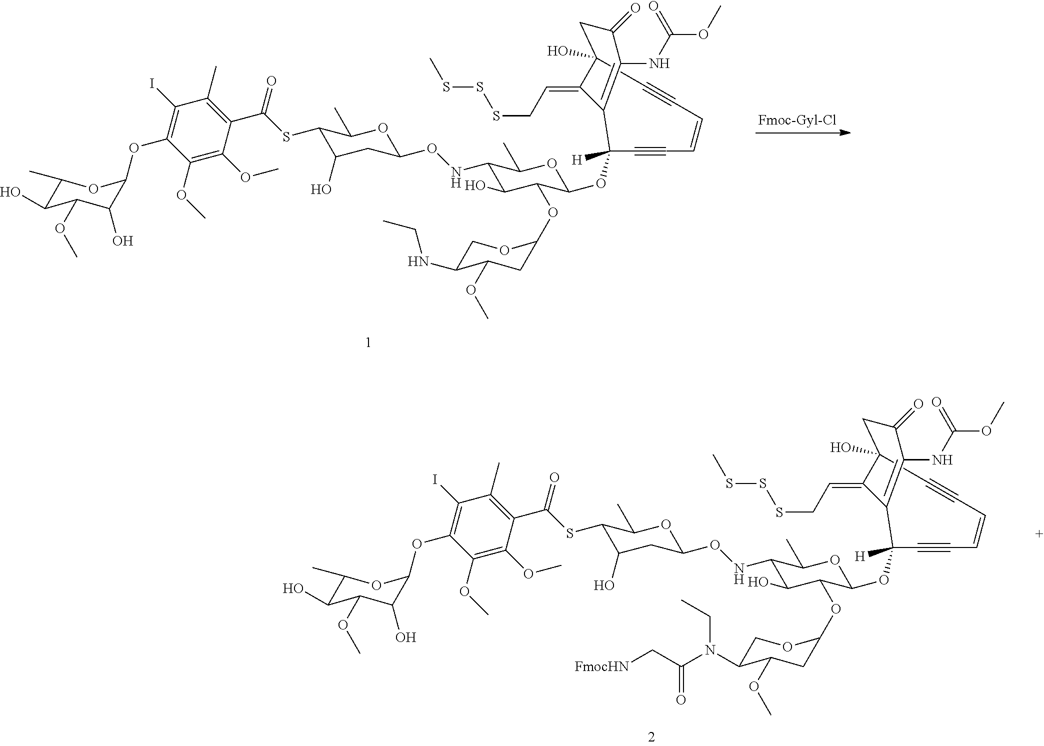

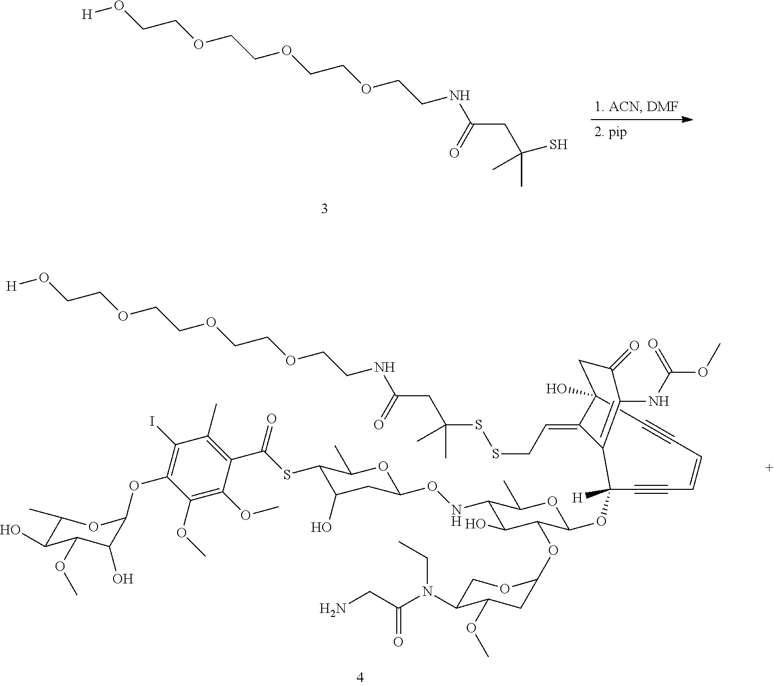 [SMILES: CC(C)(S)CC(=O)NCCOCCOCCOCCO ; CCN(C(=O)CN)C1COC(OC2C(O[C@H]3C#C/C=C\C#C[C@]4(O)CC(=O)C(NC(=O)OC)=C3/C4=C\CSSC(C)(C)CC(=O)NCCOCCOCCOCCO)OC(C)C(NOC3CC(O)C(SC(=O)c4c(C)c(I)c(OC5OC(C)C(O)C(OC)C5O)c(OC)c4OC)C(C)O3)C2O)CC1OC]

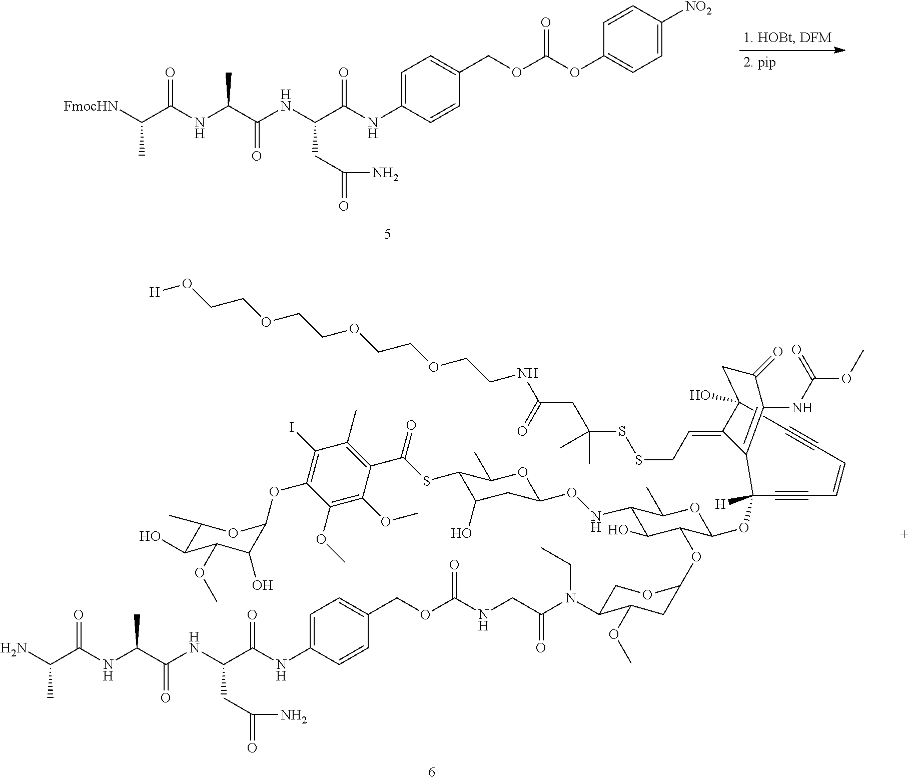

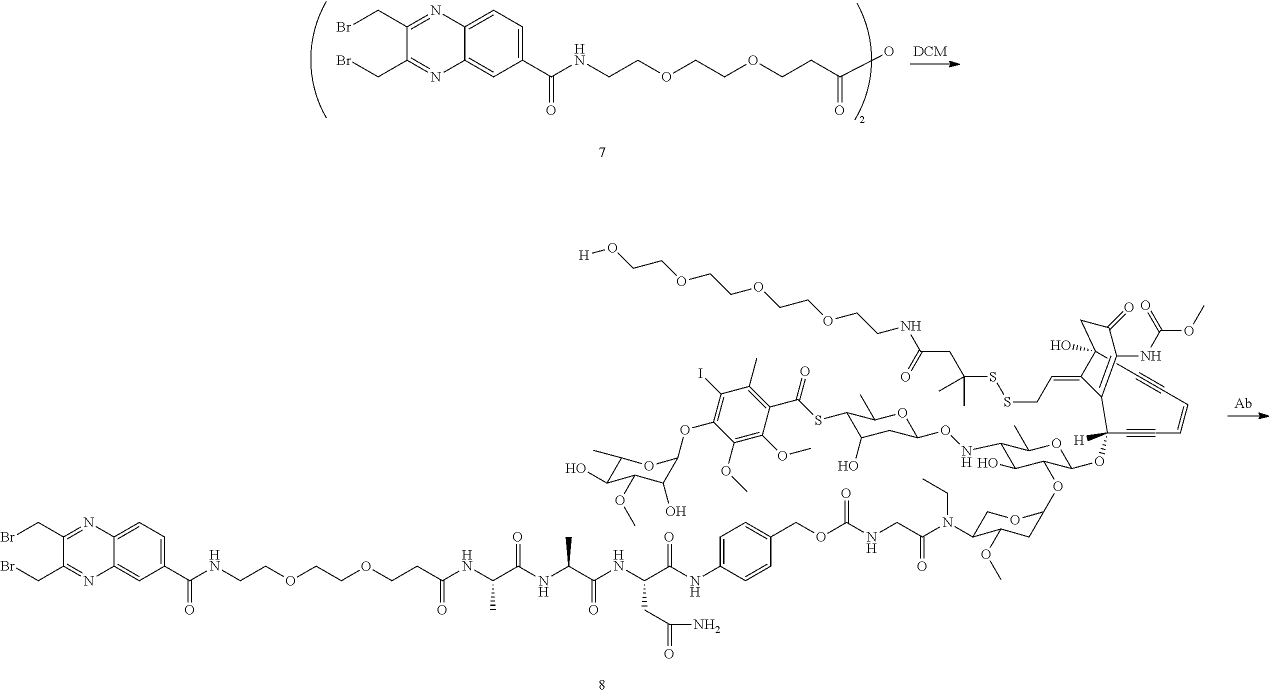

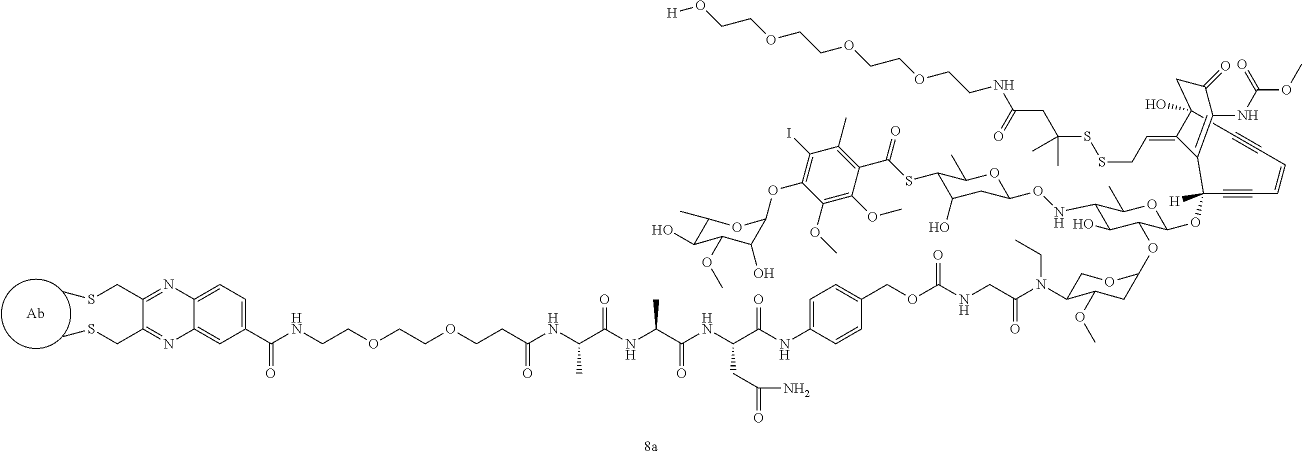

### Preparation of Compound 2:

To calicheamicin γl (1) (880 mg, 0.54 mmol) in 25 mL of dimethylformamide (DMF) was added (9H-fluoren-9-γl)methyl (2-chloro-2-oxoethyl)carbamate (256 mg, 0.81 mmol) and diisopropylethylamine (DIEA, 173 μL, 1 mmol). The mixture was stirred for 2 h, then evaporated and purified by HPLC to give compound 2 (300 mg). MS m/z 1647.3 (M+H).

### Preparation of Compound 4:

To compound 2 (100 mg, 0.06 mmol) in 4 mL of acetonitrile and 0.3 mL of DMF was added compound 3 (75 mg, 0.24 mmol). The mixture was stirred for 16 h, then 120 μL of piperidine was added. After 30 min the mixture was purified by HPLC to give compound 4 (60 mg). MS m/z 1654.4 (M+H).

### Preparation of Compound 6:

To compound 4 (20 mg, 12 μmol) in 2 mL of DMF was added compound 5 (11 mg, 14 μmol), N-Hydroxybenzotriazole (HOBt, 2 mg), and 5 μL of DIEA. The mixture was stirred for 1 h, then 40 μL of piperidine was added. After 10 min the mixture was purified by HPLC to give compound 6 (21 mg). MS m/z 2059.6 (M+H).

### Preparation of Compound 8:

To compound 6 (21 mg, 10 μmol) in 2 mL of dichloromethane (DCM) was added compound 7 (13 mg, 13 μmol), and 3 μL of DIEA. The mixture was stirred for 20 min, then evaporated and purified by HPLC to give compound 8 (11 mg). MS m/z 2558.6 (M+H).

### Preparation of Compound 10:

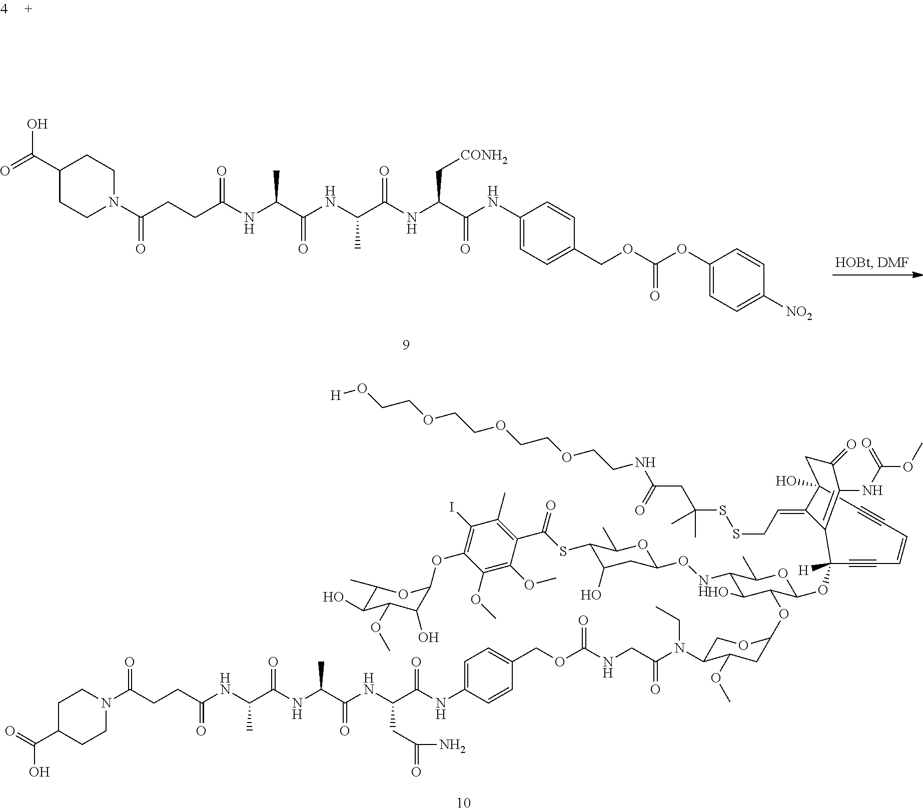 [SMILES: C[C@H](NC(=O)CCC(=O)N1CCC(C(=O)O)CC1)C(=O)N[C@@H](C)C(=O)N[C@@H](CC(N)=O)C(=O)Nc1ccc(COC(=O)Oc2ccc([N+](=O)[O-])cc2)cc1 ; CCN(C(=O)CNC(=O)OCc1ccc(NC(=O)[C@H](CC(N)=O)NC(=O)[C@H](C)NC(=O)[C@H](C)NC(=O)CCC(=O)N2CCC(C(=O)O)CC2)cc1)C1COC(OC2C(O[C@H]3C#C/C=C\C#C[C@]4(O)CC(=O)C(NC(=O)OC)=C3/C4=C\CSSC(C)(C)CC(=O)NCCOCCOCCOCCO)OC(C)C(NOC3CC(O)C(SC(=O)c4c(C)c(I)c(OC5OC(C)C(O)C(OC)C5O)c(OC)c4OC)C(C)O3)C2O)CC1OC]

To compound 4 (12 mg, 7.3 μmol) in 1 mL of DMF was added compound 9 (7.1 mg, 9.4 μmol), N-Hydroxybenzotriazole (1 mg), and 5 μL of DIEA. The mixture was stirred for 1 h, then purified by HPLC to give compound 10 (12 mg). MS m/z 2270.6 (M+H).

### Preparation of Compound 14 and 14a:

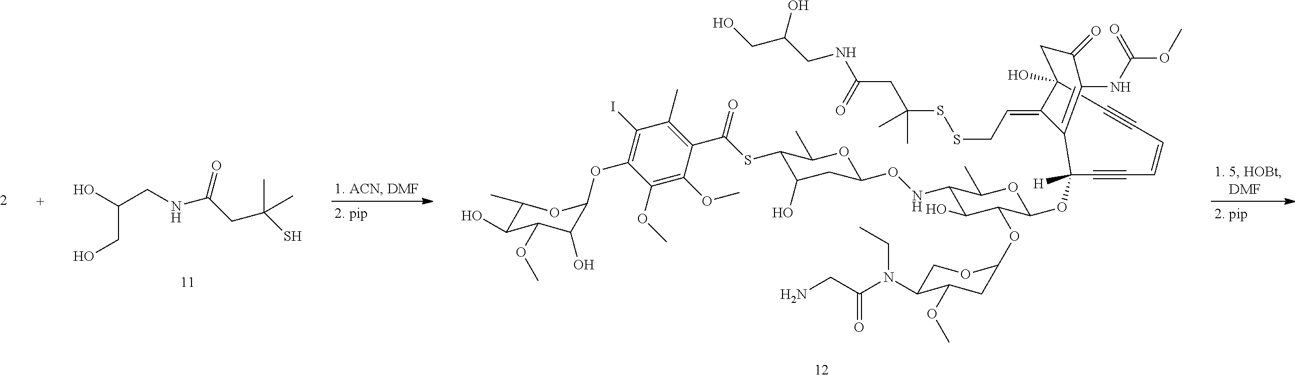 [SMILES: CC(C)(S)CC(=O)NCC(O)CO ; CCN(C(=O)CN)C1COC(OC2C(O[C@H]3C#C/C=C\C#C[C@]4(O)CC(=O)C(NC(=O)OC)=C3/C4=C\CSSC(C)(C)CC(=O)NCC(O)CO)OC(C)C(NOC3CC(O)C(SC(=O)c4c(C)c(I)c(OC5OC(C)C(O)C(OC)C5O)c(OC)c4OC)C(C)O3)C2O)CC1OC]

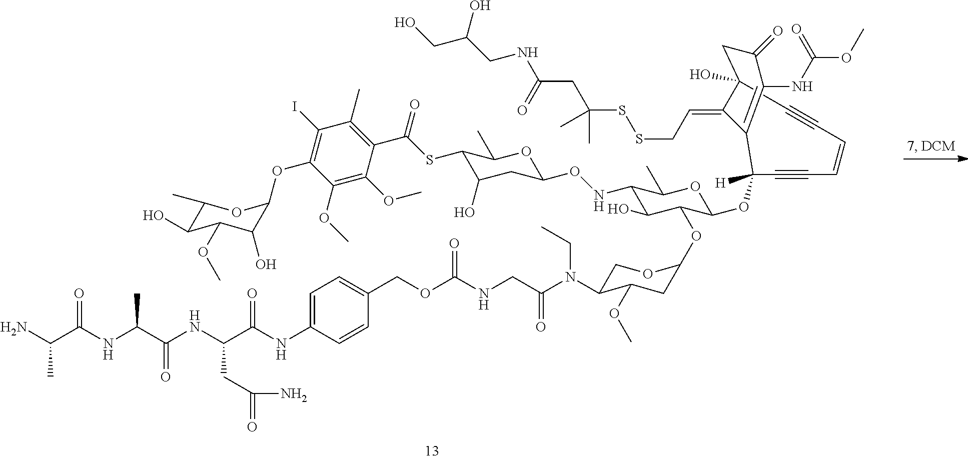 [SMILES: CCN(C(=O)CNC(=O)OCc1ccc(NC(=O)[C@H](CC(N)=O)NC(=O)[C@H](C)NC(=O)[C@H](C)N)cc1)C1COC(OC2C(O[C@H]3C#C/C=C\C#C[C@]4(O)CC(=O)C(NC(=O)OC)=C3/C4=C\CSSC(C)(C)CC(=O)NCC(O)CO)OC(C)C(NOC3CC(O)C(SC(=O)c4c(C)c(I)c(OC5OC(C)C(O)C(OC)C5O)c(OC)c4OC)C(C)O3)C2O)CC1OC]

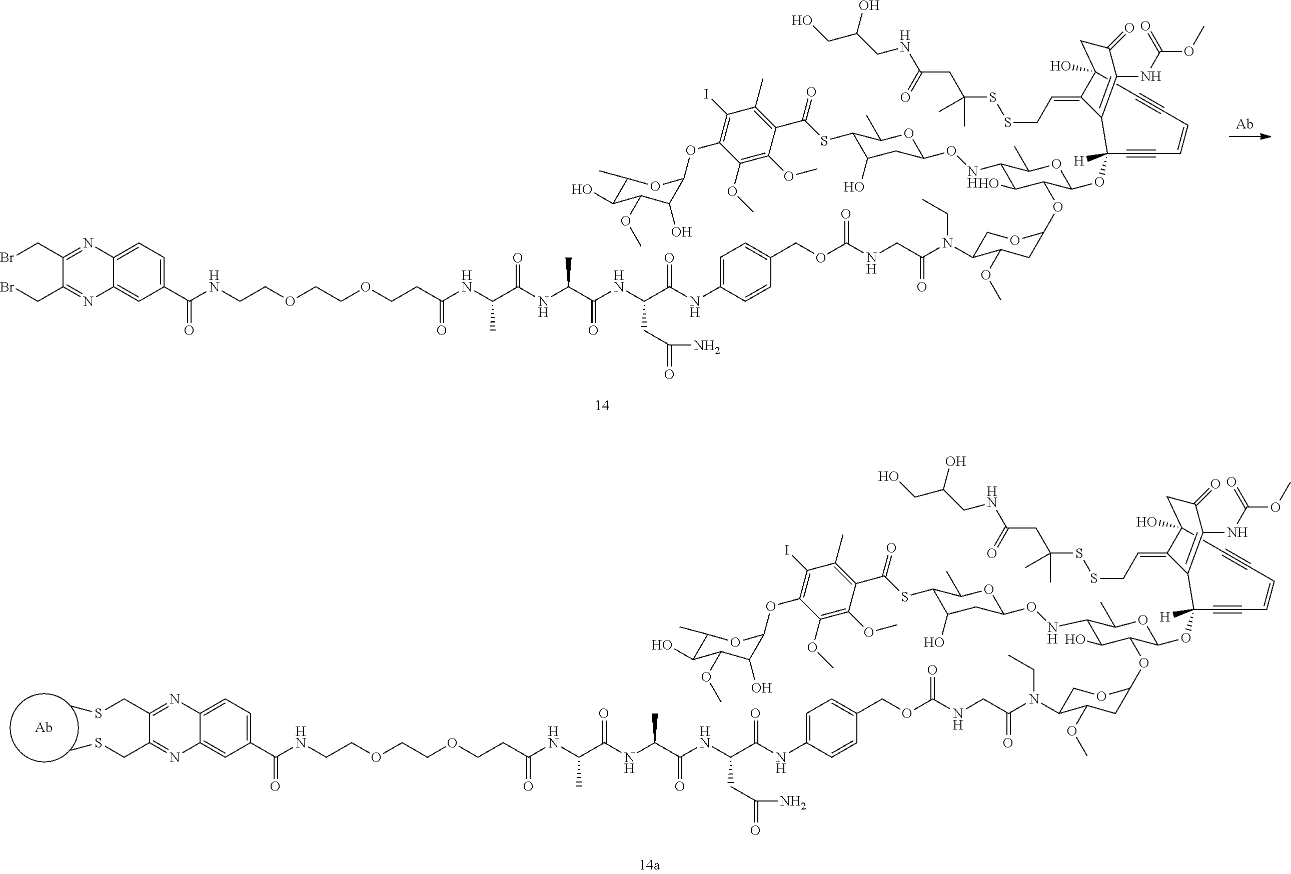

### Preparation of Compound 12:

To compound 2 (100 mg, 0.06 mmol) in 4 mL of acetonitrile and 0.3 mL of DMF was added compound 11 (118 mg, 0.57 mmol). The mixture was stirred for 16 h, then 120 μL of piperidine was added. After 30 min the mixture was purified by HPLC to give compound 12 (40 mg). MS m/z 1552.4 (M+H).

### Preparation of Compound 13:

To compound 12 (28 mg, 18 mmol) in 1 mL of DMF was added compound 5 (17 mg, 22 mmol), HOBt (2 mg), and 5 μL of DIEA. The mixture was stirred for 1 h, then 20 μL of piperidine was added. After 10 min the mixture was purified by HPLC to give compound 13 (23 mg). MS m/z 1957.6 (M+H).

### Preparation of Compound 14:

To compound 13 (23 mg, 12 μmol) in 2 mL of DCM was added compound 7 (17 mg, 17 μmol), and 3 μL of DIEA. The mixture was stirred for 20 min, then evaporated and purified by HPLC to give compound 14 (16 mg). MS m/z 2558.6 (M+H).

### Preparation of Compound 15:

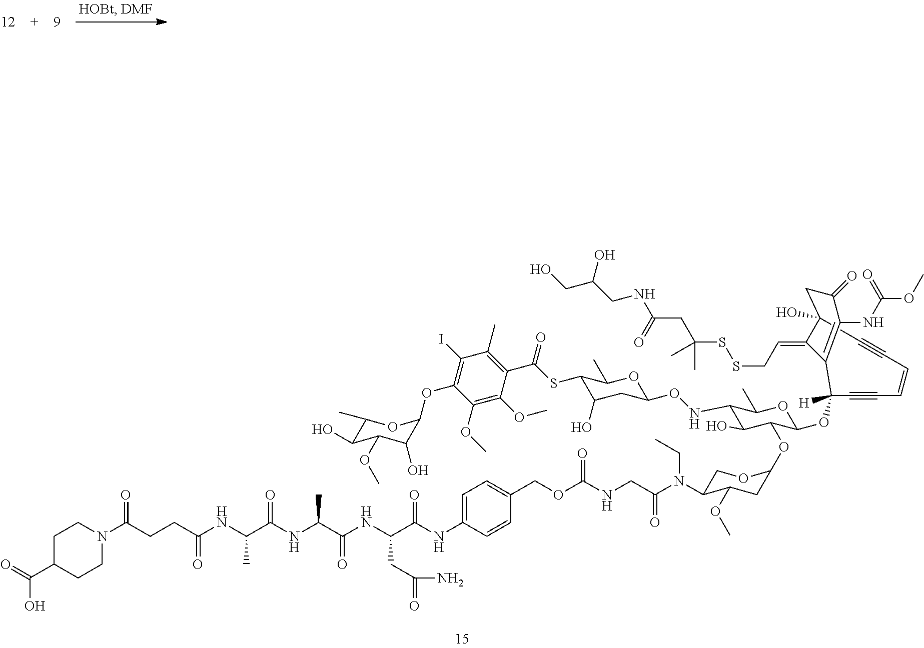 [SMILES: CCN(C(=O)CNC(=O)OCc1ccc(NC(=O)[C@H](CC(N)=O)NC(=O)[C@H](C)NC(=O)[C@H](C)NC(=O)CCC(=O)N2CCC(C(=O)O)CC2)cc1)C1COC(OC2C(O[C@H]3C#C/C=C\C#C[C@]4(O)CC(=O)C(NC(=O)OC)=C3/C4=C\CSSC(C)(C)CC(=O)NCC(O)CO)OC(C)C(NOC3CC(O)C(SC(=O)c4c(C)c(I)c(OC5OC(C)C(O)C(OC)C5O)c(OC)c4OC)C(C)O3)C2O)CC1OC]

To compound 12 (10 mg, 6.5 μmol) in 1 mL of DMF was added compound 9 (7.1 mg, 9.4 μmol), HOBt (1 mg), and 5 μL of DIEA. The mixture was stirred for 1 h, then purified by HPLC to give compound 15 (11 mg). MS m/z 2168.6 (M+H).

### Preparation of Compound 18 and 18a:

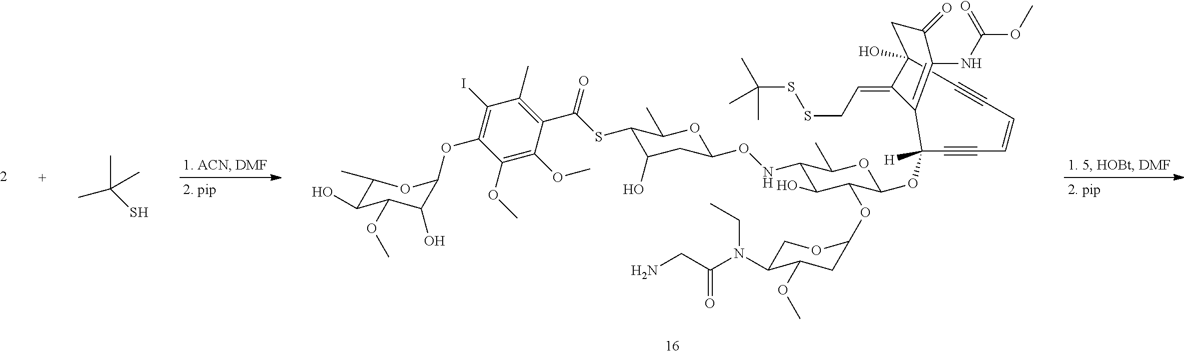

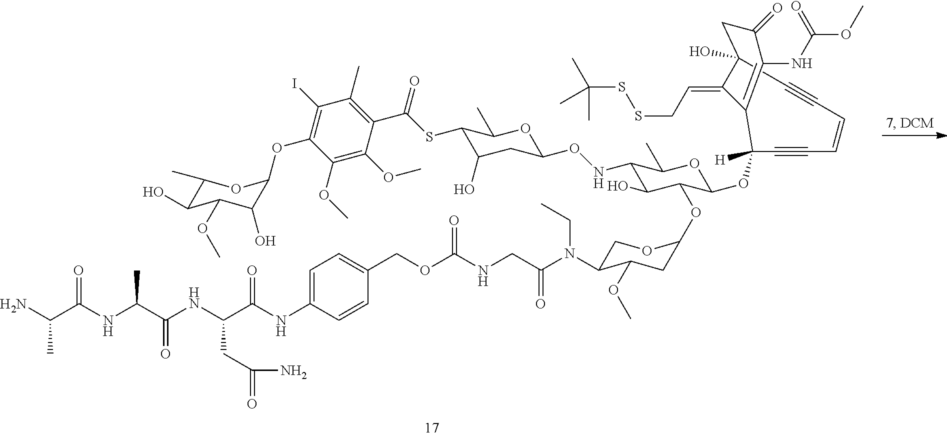 [SMILES: CCN(C(=O)CNC(=O)OCc1ccc(NC(=O)[C@H](CC(N)=O)NC(=O)[C@H](C)NC(=O)[C@H](C)N)cc1)C1COC(OC2C(O[C@H]3C#C/C=C\C#C[C@]4(O)CC(=O)C(NC(=O)OC)=C3/C4=C\CSSC(C)(C)C)OC(C)C(NOC3CC(O)C(SC(=O)c4c(C)c(I)c(OC5OC(C)C(O)C(OC)C5O)c(OC)c4OC)C(C)O3)C2O)CC1OC]

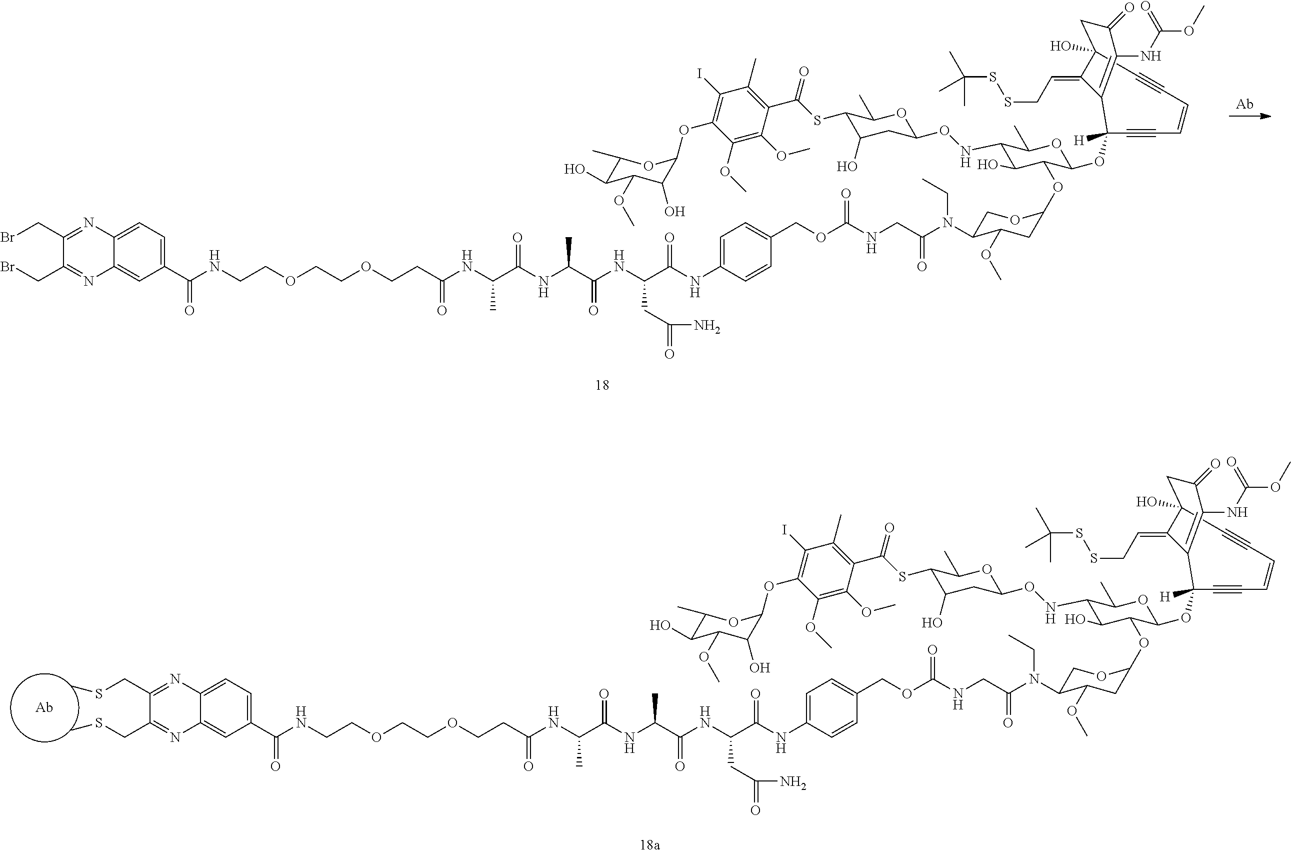

### Preparation of Compound 16:

To compound 2 (100 mg, 0.06 mmol) in 4 mL of acetonitrile and 0.3 mL of DMF was added tert-butyl thiol (54 mg, 0.6 mmol). The mixture was stirred for 16 h, then 120 μL of piperidine was added. After 30 min the mixture was purified by HPLC to give compound 16 (55 mg). MS m/z 1435.4 (M+H).

### Preparation of Compound 17:

To compound 16 (20 mg, 14 μmol) in 2 mL of DMF was added compound 5 (13 mg, 17 μmol), HOBt (2 mg), and 5 μL of DIEA. The mixture was stirred for 1 h, then 40 μL of piperidine was added. After 10 min the mixture was purified by HPLC to give compound 17 (20 mg). MS m/z 1840.6 (M+H).

### Preparation of Compound 18:

To compound 17 (20 mg, 11 μmol) in 2 mL of DCM was added compound 7 (14 mg, 14 μmol), and 3 μL of DIEA. The mixture was stirred for 20 min, then evaporated and purified by HPLC to give compound 18 (16 mg). MS m/z 2339.6 (M+H).

### Preparation of Compound 19:

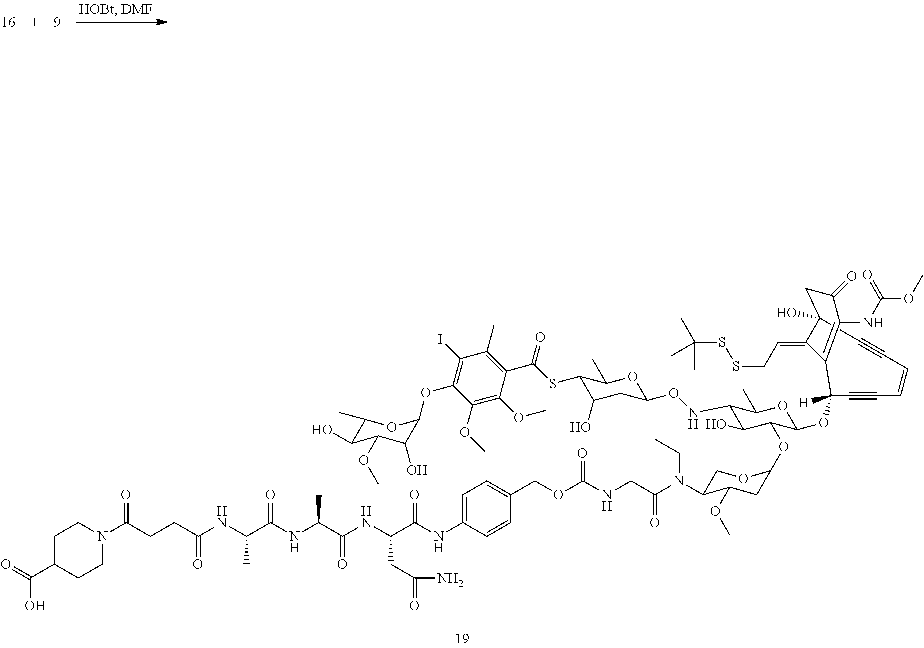 [SMILES: CCN(C(=O)CNC(=O)OCc1ccc(NC(=O)[C@H](CC(N)=O)NC(=O)[C@H](C)NC(=O)[C@H](C)NC(=O)CCC(=O)N2CCC(C(=O)O)CC2)cc1)C1COC(OC2C(O[C@H]3C#C/C=C\C#C[C@]4(O)CC(=O)C(NC(=O)OC)=C3/C4=C\CSSC(C)(C)C)OC(C)C(NOC3CC(O)C(SC(=O)c4c(C)c(I)c(OC5OC(C)C(O)C(OC)C5O)c(OC)c4OC)C(C)O3)C2O)CC1OC]

To compound 16 (23 mg, 16 μmol) in 1 mL of DMF was added compound 9 (13 mg, 17 μmol), HOBt (2 mg), and 7 μL of DIEA. The mixture was stirred for 5 h, then purified by HPLC to give compound 19 (21 mg). MS m/z 2051.6 (M+H).

### Preparation of Compound 23 and 23a:

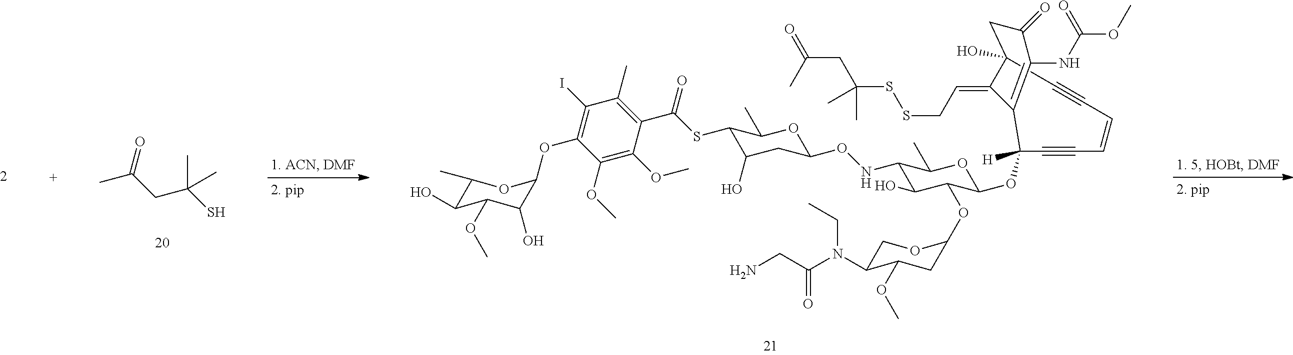

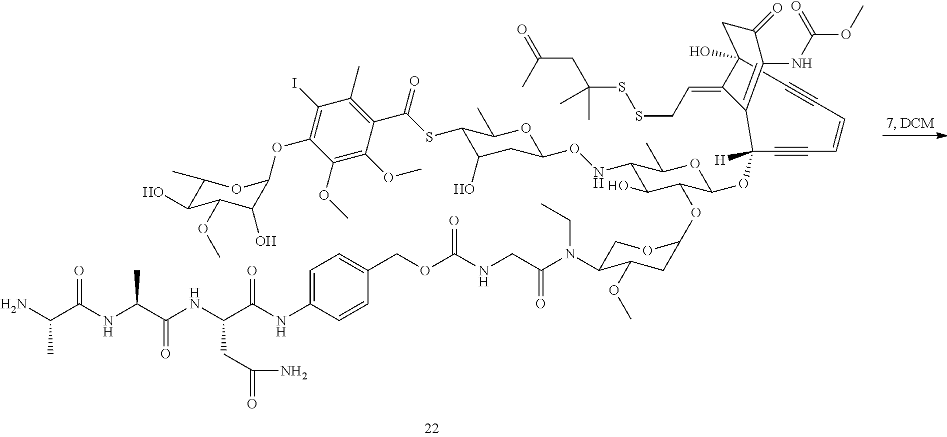 [SMILES: CCN(C(=O)CNC(=O)OCc1ccc(NC(=O)[C@H](CC(N)=O)NC(=O)[C@H](C)NC(=O)[C@H](C)N)cc1)C1COC(OC2C(O[C@H]3C#C/C=C\C#C[C@]4(O)CC(=O)C(NC(=O)OC)=C3/C4=C\CSSC(C)(C)CC(C)=O)OC(C)C(NOC3CC(O)C(SC(=O)c4c(C)c(I)c(OC5OC(C)C(O)C(OC)C5O)c(OC)c4OC)C(C)O3)C2O)CC1OC]

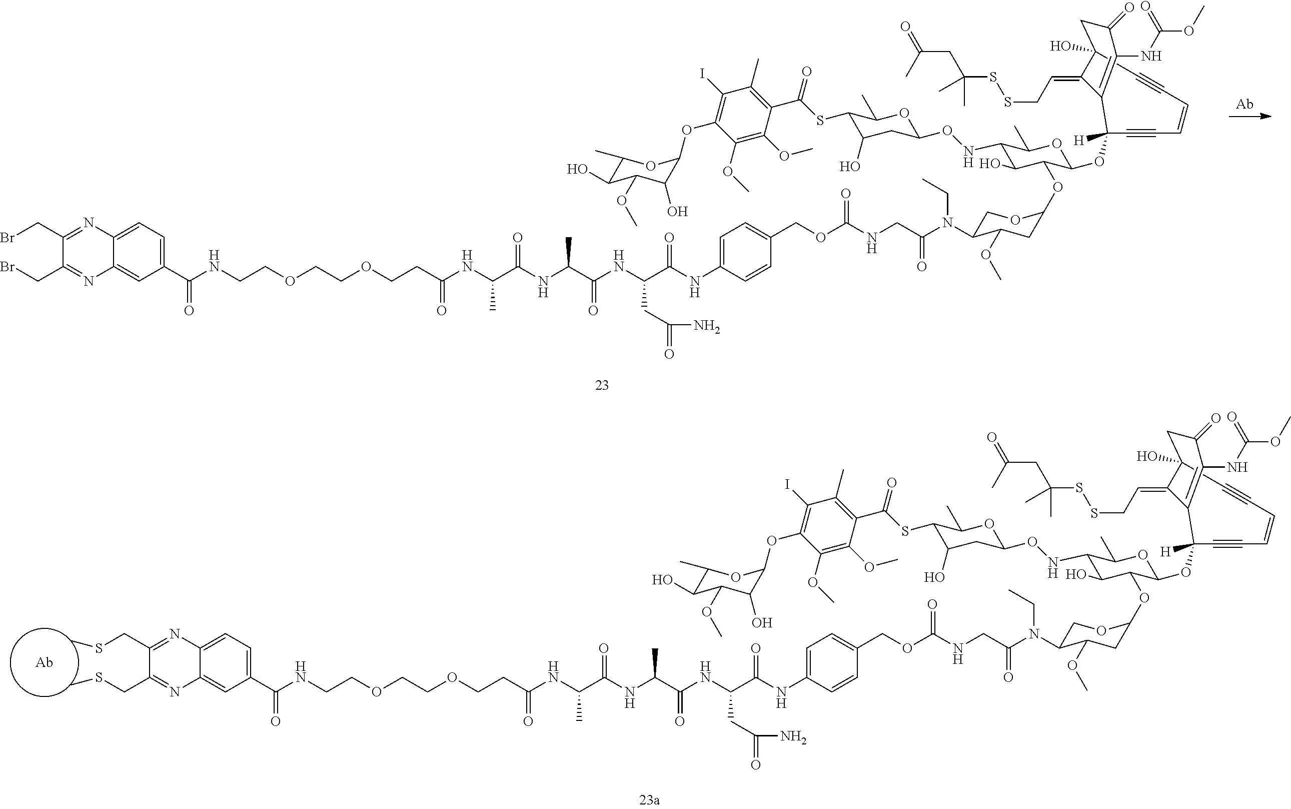

### Preparation of Compound 21:

To compound 2 (100 mg, 0.06 mmol) in 4 mL of acetonitrile and 0.3 mL of DMF was added compound 20 (24 mg, 0.18 mmol). The mixture was stirred for 16 h, then 120 μL of piperidine was added. After 30 min the mixture was purified by HPLC to give compound 21(50 mg). MS m/z 1477.4 (M+H).

### Preparation of Compound 22:

To compound 21 (20 mg, 14 μmol) in 2 mL of DMF was added compound 5 (13 mg, 17 μmol), HOBt (2 mg), and 5 μL of DIEA. The mixture was stirred for 1 h, then 40 μL of piperidine was added. After 10 min the mixture was purified by HPLC to give compound 22 (8 mg). MS m/z 1882.6 (M+H).

### Preparation of Compound 23:

To compound 22 (8 mg, 4 μmol) in 2 mL of DCM was added compound 7 (6 mg, 6 μmol), and 3 μL of DIEA. The mixture was stirred for 20 min, then evaporated and purified by HPLC to give compound 23 (6 mg). MS m/z 2381.6 (M+H).

### Preparation of Compound 24:

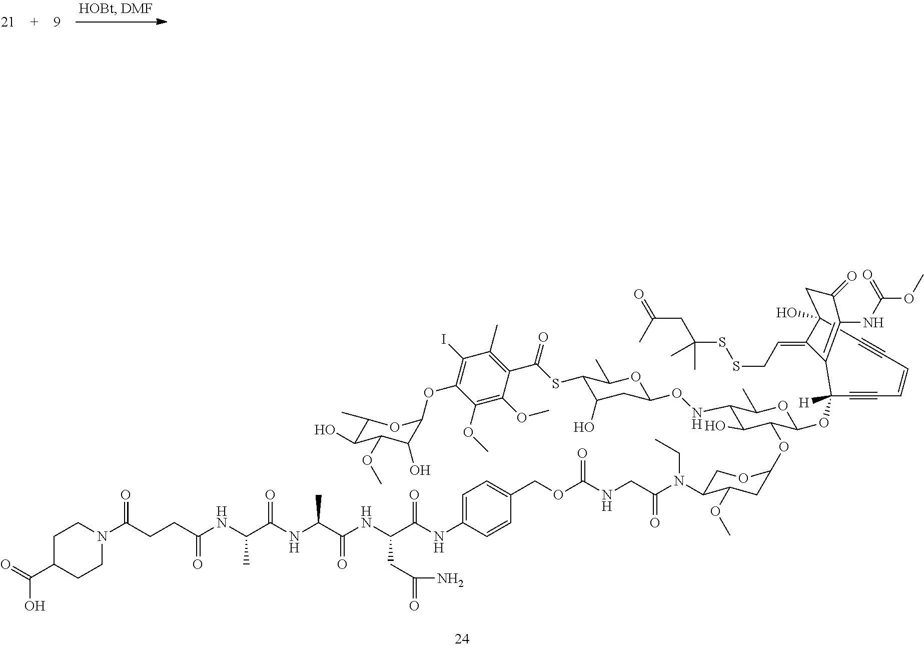 [SMILES: CCN(C(=O)CNC(=O)OCc1ccc(NC(=O)[C@H](CC(N)=O)NC(=O)[C@H](C)NC(=O)[C@H](C)NC(=O)CCC(=O)N2CCC(C(=O)O)CC2)cc1)C1COC(OC2C(O[C@H]3C#C/C=C\C#C[C@]4(O)CC(=O)C(NC(=O)OC)=C3/C4=C\CSSC(C)(C)CC(C)=O)OC(C)C(NOC3CC(O)C(SC(=O)c4c(C)c(I)c(OC5OC(C)C(O)C(OC)C5O)c(OC)c4OC)C(C)O3)C2O)CC1OC]

To compound 16 (23 mg, 16 μmol) in 1 mL of DMF was added compound 9 (13 mg, 17 μmol), HOBt (2 mg), and 7 μL of DIEA. The mixture was stirred for 5 h, then purified by HPLC to give compound 19 (21 mg). MS m/z 2093.6 (M+H).

### Preparation of Compound 30 and 30a:

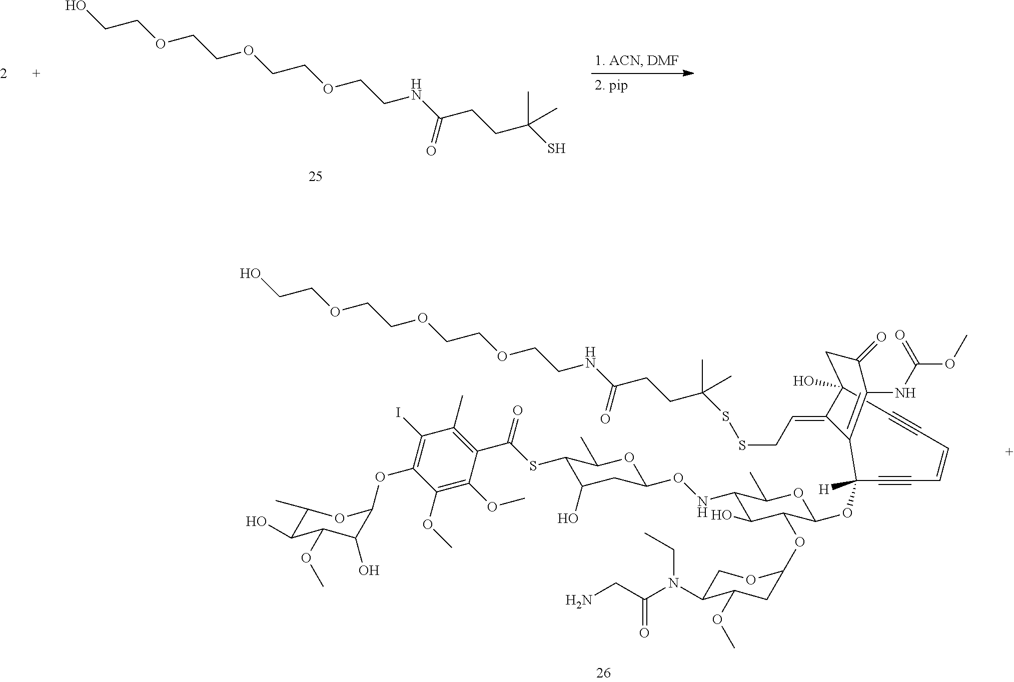 [SMILES: CC(C)(S)CCC(=O)NCCOCCOCCOCCO ; CCN(C(=O)CN)C1COC(OC2C(O[C@H]3C#C/C=C\C#C[C@]4(O)CC(=O)C(NC(=O)OC)=C3/C4=C\CSSC(C)(C)CCC(=O)NCCOCCOCCOCCO)OC(C)C(NOC3CC(O)C(SC(=O)c4c(C)c(I)c(OC5OC(C)C(O)C(OC)C5O)c(OC)c4OC)C(C)O3)C2O)CC1OC]

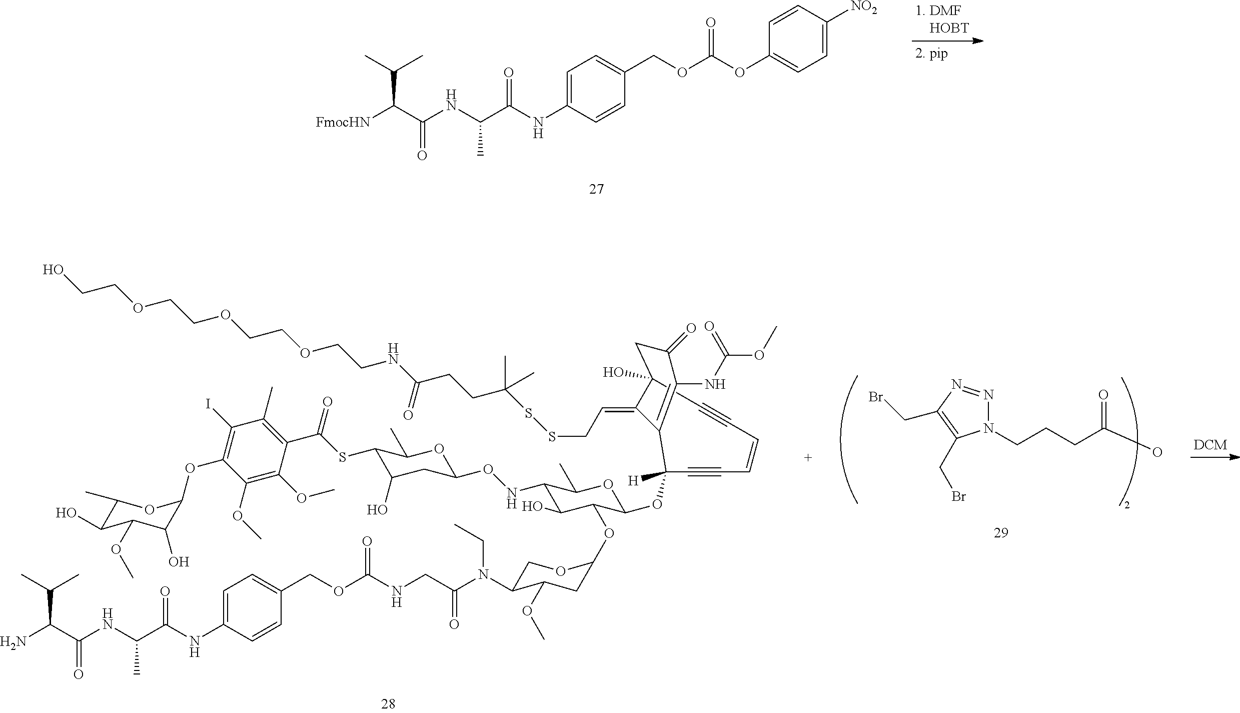

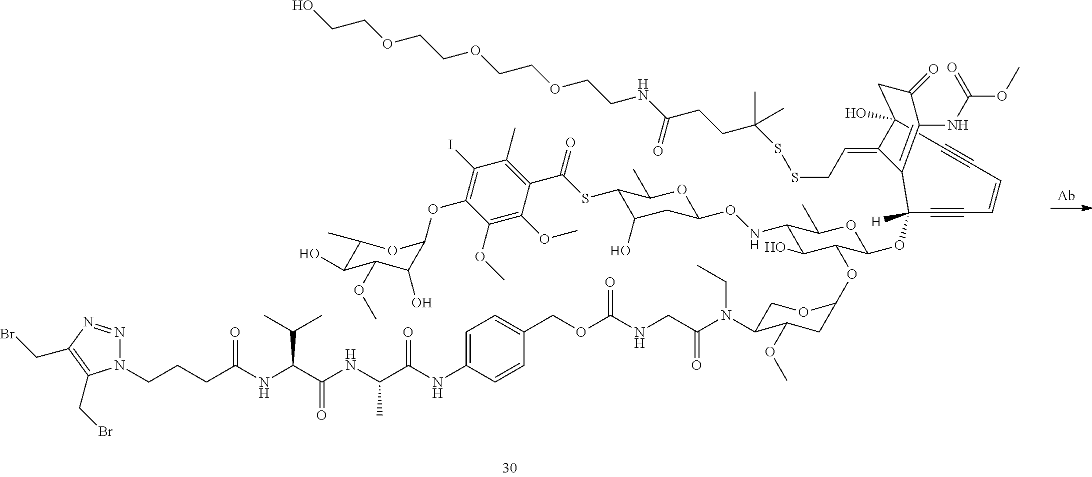 [SMILES: CCN(C(=O)CNC(=O)OCc1ccc(NC(=O)[C@H](C)NC(=O)[C@@H](NC(=O)CCCn2nnc(CBr)c2CBr)C(C)C)cc1)C1COC(OC2C(O[C@H]3C#C/C=C\C#C[C@]4(O)CC(=O)C(NC(=O)OC)=C3/C4=C\CSSC(C)(C)CCC(=O)NCCOCCOCCOCCO)OC(C)C(NOC3CC(O)C(SC(=O)c4c(C)c(I)c(OC5OC(C)C(O)C(OC)C5O)c(OC)c4OC)C(C)O3)C2O)CC1OC]

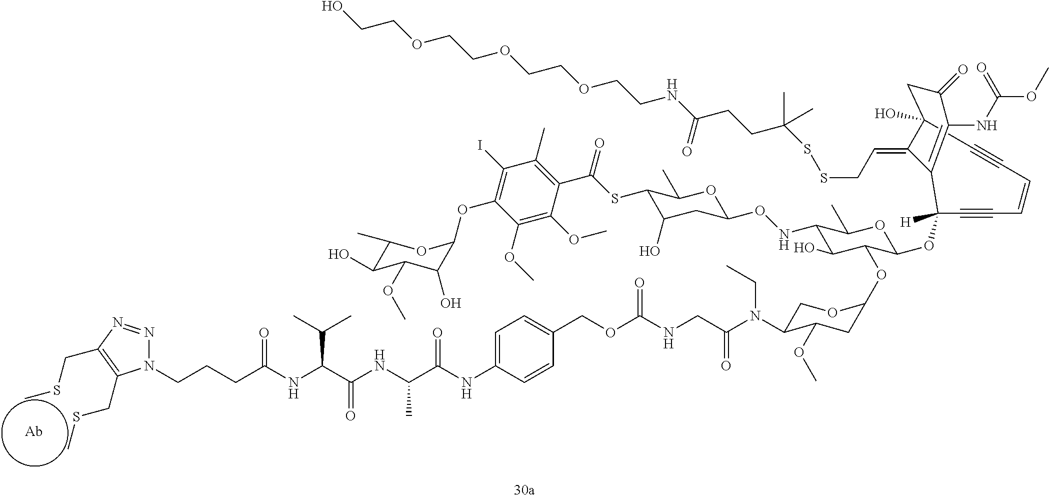

### Preparation of Compound 26:

To compound 2 (313 mg, 0.19 mmol) in 3 mL of acetonitrile and 1 mL of DMF was added compound 25 (184 mg, 0.57 mmol). The mixture was stirred for 16 h, then 120 μL of piperidine was added. After 20 min the mixture was purified by HPLC to give compound 26 (160 mg). MS m/z 1668.4 (M+H).

### Preparation of Compound 28:

To compound 26 (140 mg, 84 μmol) in 3 mL of DMF was added compound 27 (69 mg, 101 μmol), HOBt (11 mg), and 43 μL of DIEA. The mixture was stirred for 1 h, then 150 μL of piperidine was added. After 10 min the mixture was purified by HPLC to give compound 28 (112 mg). MS m/z 1987.6 (M+H).

### Preparation of Compound 30:

To compound 28 (22 mg, 11 μmol) in 2 mL of DCM was added compound 29 (8.6 mg, 13 μmol), and 3 μL of DIEA. The mixture was stirred for 20 min, then evaporated and purified by HPLC to give compound 30 (12 mg). MS m/z 2308.6 (M+H).

### Preparation of Compound 34:

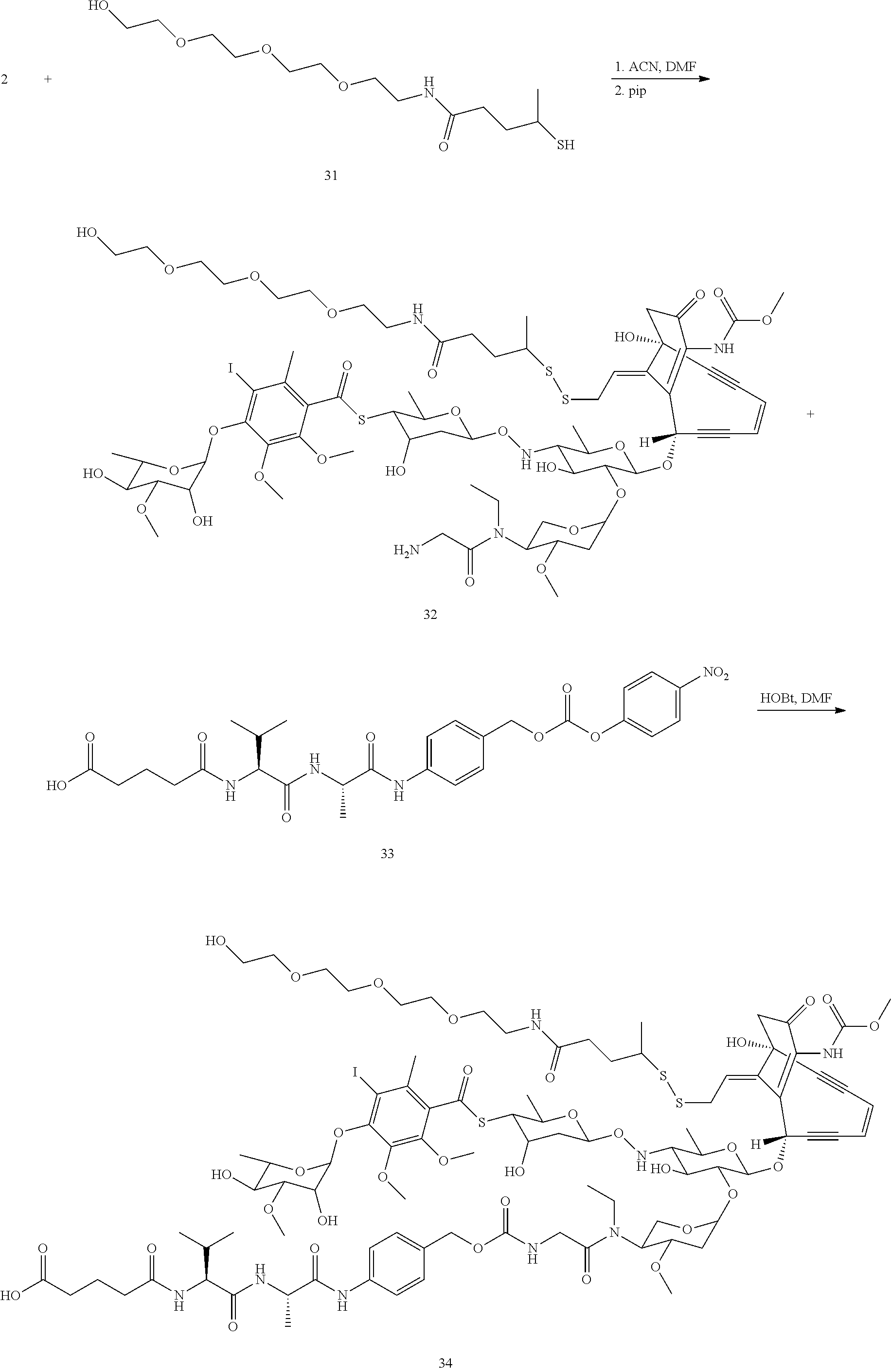

### Preparation of Compound 32:

To compound 2 (100 mg, 0.06 mmol) in 4 mL of acetonitrile and 0.3 mL of DMF was added compound 31 (75 mg, 0.24 mmol). The mixture was stirred for 16 h, then 120 μL of piperidine was added. After 30 min the mixture was purified by HPLC to give compound 32 (50 mg). MS m/z 1654.4 (M+H).

### Preparation of Compound 34:

To compound 32 (20 mg, 12 μmol) in 1 mL of DMF was added compound 33 (10 mg, 17 μmol), HOBt (2 mg), and 5 μL of DIEA. The mixture was stirred for 5 h, then purified by

HPLC to give compound 34 (18 mg). MS m/z 2087.6 (M+H).

### Preparation of Compound 37:

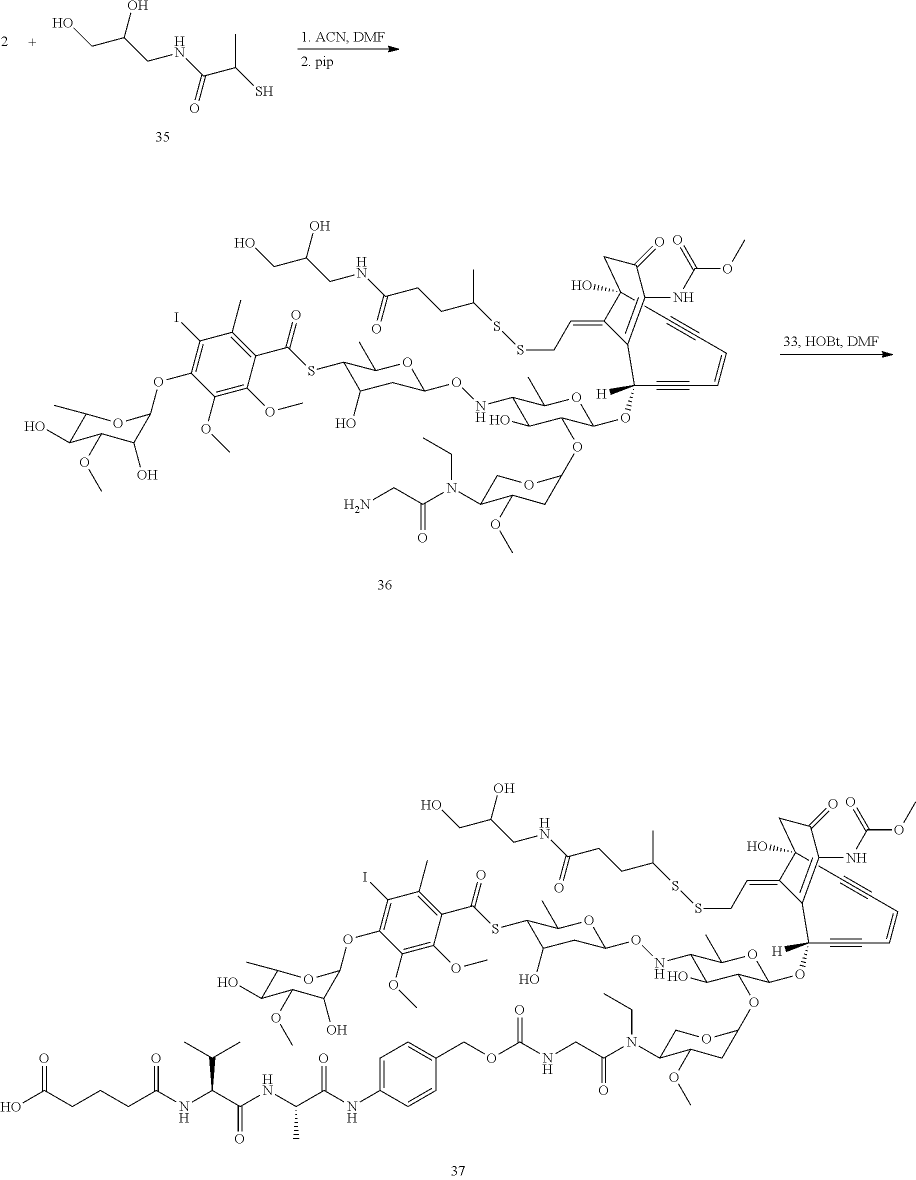 [SMILES: CC(S)C(=O)NCC(O)CO ; CCN(C(=O)CN)C1COC(OC2C(O[C@H]3C#C/C=C\C#C[C@]4(O)CC(=O)C(NC(=O)OC)=C3/C4=C\CSSC(C)CCC(=O)NCC(O)CO)OC(C)C(NOC3CC(O)C(SC(=O)c4c(C)c(I)c(OC5OC(C)C(O)C(OC)C5O)c(OC)c4OC)C(C)O3)C2O)CC1OC ; CCN(C(=O)CNC(=O)OCc1ccc(NC(=O)[C@H](C)NC(=O)[C@@H](NC(=O)CCCC(=O)O)C(C)C)cc1)C1COC(OC2C(O[C@H]3C#C/C=C\C#C[C@]4(O)CC(=O)C(NC(=O)OC)=C3/C4=C\CSSC(C)CCC(=O)NCC(O)CO)OC(C)C(NOC3CC(O)C(SC(=O)c4c(C)c(I)c(OC5OC(C)C(O)C(OC)C5O)c(OC)c4OC)C(C)O3)C2O)CC1OC]

### Preparation of Compound 36:

To compound 2 (100 mg, 0.06 mmol) in 4 mL of acetonitrile and 0.3 mL of DMF was added compound 35 (37 mg, 0.18 mmol). The mixture was stirred for 16 h, then 120 μL of piperidine was added. After 30 min the mixture was purified by HPLC to give compound 36 (45 mg). MS m/z 1552.4 (M+H).

### Preparation of Compound 37:

To compound 32 (20 mg, 13 μmol) in 1 mL of DMF was added compound 33 (10 mg, 17 μmol) HOBt (2 mg), and 5 μL of DIEA. The mixture was stirred for 5 h, then purified by HPLC to give compound 37 (19 mg). MS m/z 1985.6 (M+H).

### Preparation of Compound 40:

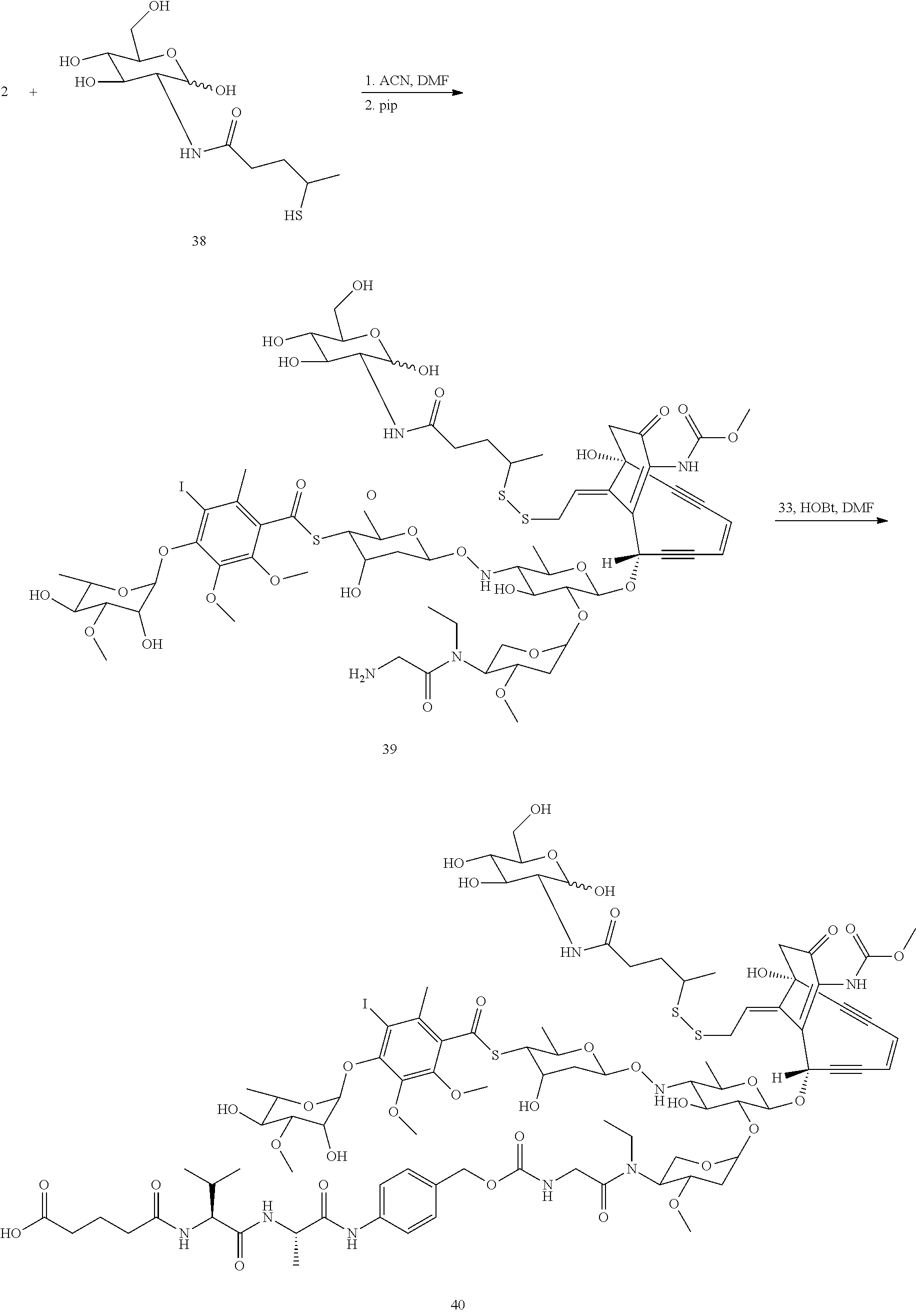

### Preparation of Compound 39:

To compound 2 (100 mg, 0.06 mmol) in 4 mL of acetonitrile and 0.3 mL of DMF was added compound 38 (53 mg, 0.18 mmol). The mixture was stirred for 16 h, then 120 μL of piperidine was added. After 30 min the mixture was purified by HPLC to give compound 39 (43 mg). MS m/z 1640.4 (M+H).

### Preparation of Compound 40:

To compound 39 (20 mg, 12 μmol) in 1 mL of DMF was added compound 33 (10 mg, 17 μmol), HOBt (2 mg), and 5 μL of DIEA. The mixture was stirred for 5 h, then purified by HPLC to give compound 40 (16 mg). MS m/z 2073.6 (M+H).

### Preparation of Compound 44:

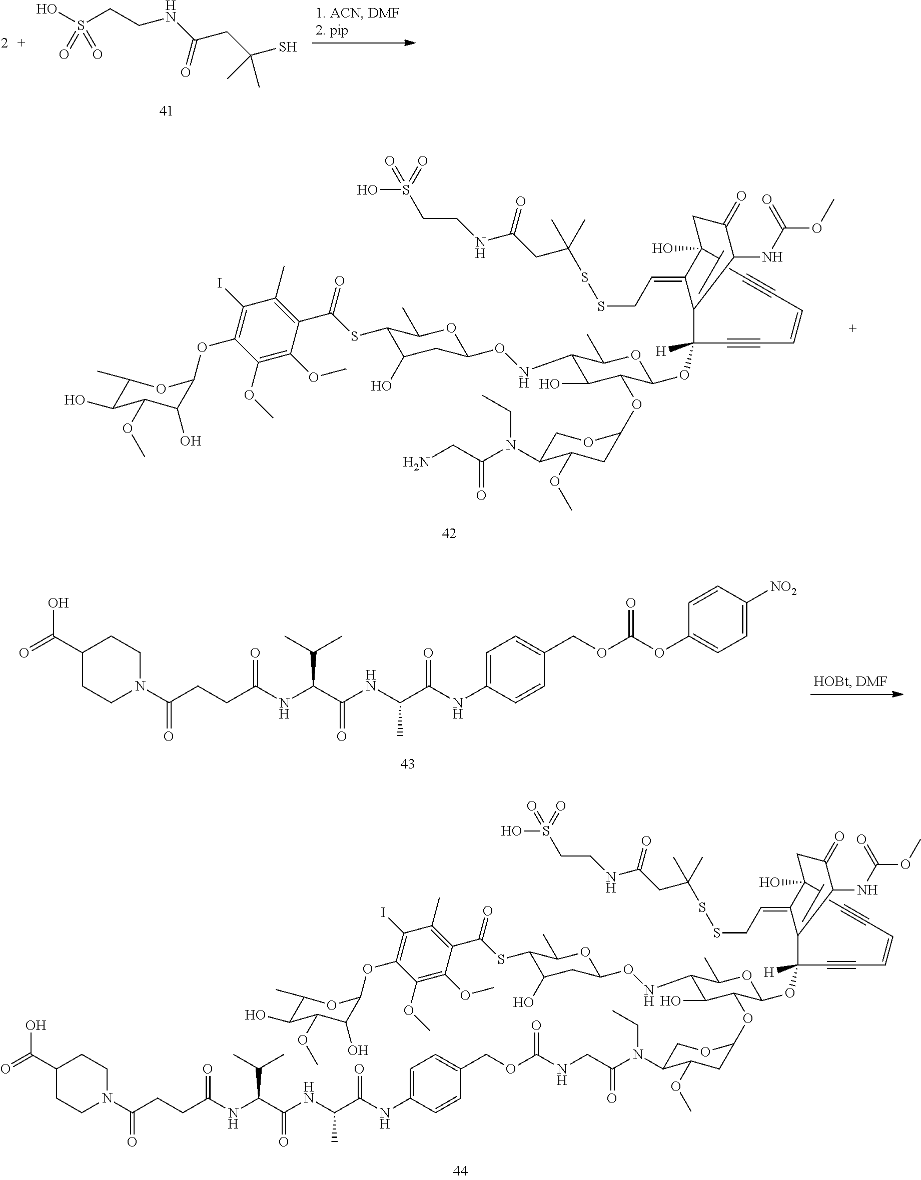

### Preparation of Compound 42:

To compound 2 (100 mg, 0.06 mmol) in 4 mL of acetonitrile and 0.3 mL of DMF was added compound 41 (88 mg, 0.36 mmol). The mixture was stirred for 16 h, then 120 μL of piperidine was added. After 30 min the mixture was purified by HPLC to give compound 42 (53 mg). MS m/z 1586.4 (M+H).

### Preparation of Compound 44:

To compound 42 (19 mg, 12 μmol) in 1 mL of DMF was added compound 43 (10 mg, 15 μmol), HOBt (2 mg), and 8 μL of DIEA. The mixture was stirred for 5 h, then purified by HPLC to give compound 44 (25 mg). MS m/z 2116.6 (M+H).

### Preparation of Compound 46:

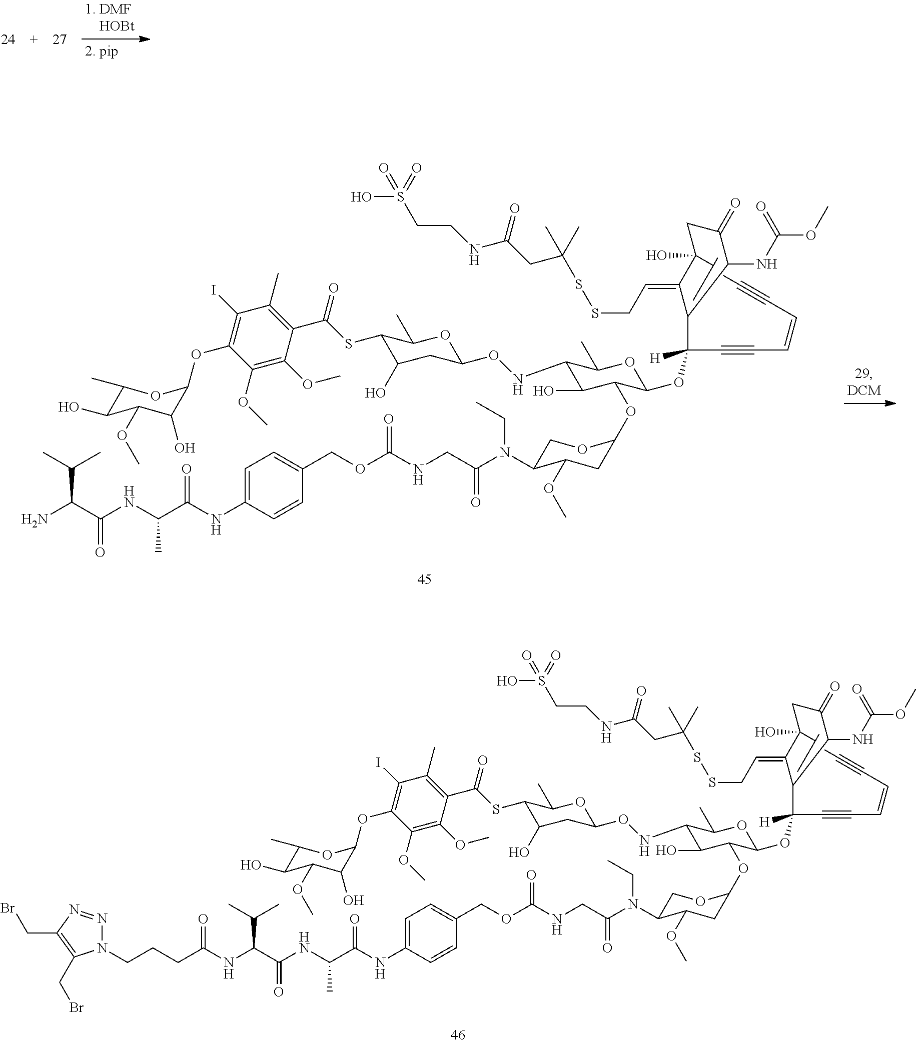

### Preparation of Compound 45:

To compound 42 (30 mg, 19 umol) in 2 mL of DMF was added compound 27 (16 mg, 23 μmol), HOBt (5 mg), and 13 μL of DIEA. The mixture was stirred for 1 h, then 40 μL of piperidine was added. After 10 min the mixture was purified by HPLC to give compound 45 (22 mg). MS m/z 1905.5 (M+H).

### Preparation of Compound 46:

To compound 45 (18 mg, 10 μmol) in 2 mL of DCM was added compound 29 (8.6 mg, 13 μmol), and 3 μL of DIEA. The mixture was stirred for 20 min, then evaporated and purified by HPLC to give compound 46 (20 mg). MS m/z 2226.6 (M+H).

### Preparation of Compound 48:

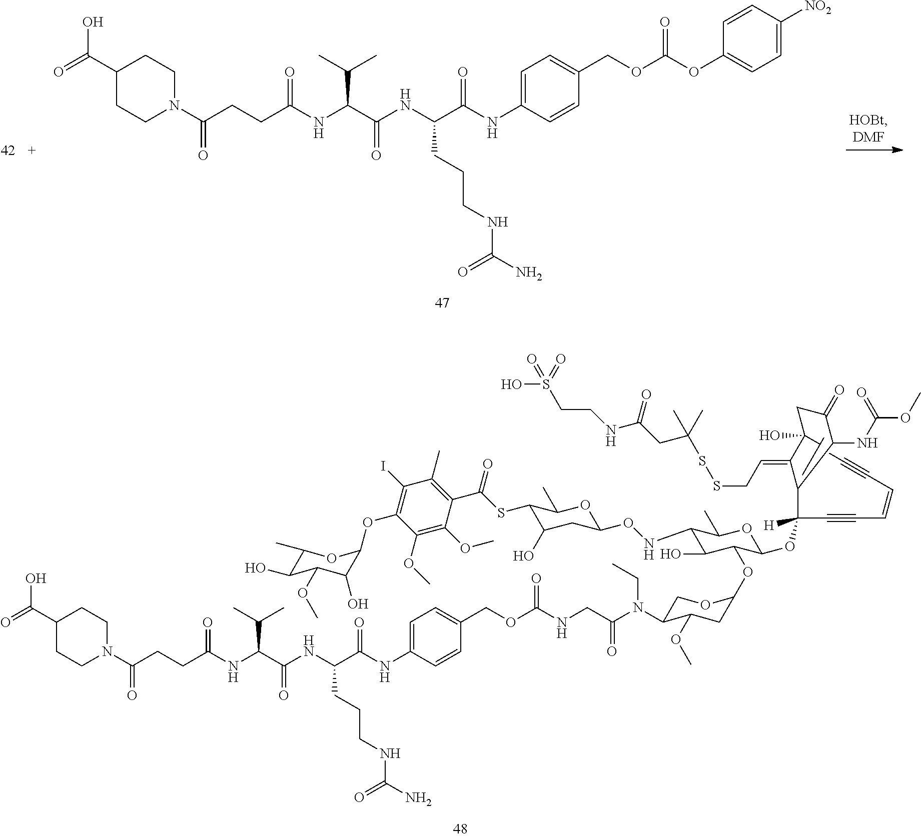

To compound 42 (6.2 mg, 3.9 μmol) in 1 mL of DMF was added compound 47 (3.1 mg, 4.2 μmol), HOBt (1 mg), and 3 μL of DIEA. The mixture was stirred for 1 h, then purified by HPLC to give compound 48 (5 mg). MS m/z 2202.6 (M+H).

### Preparation of Compound 51:

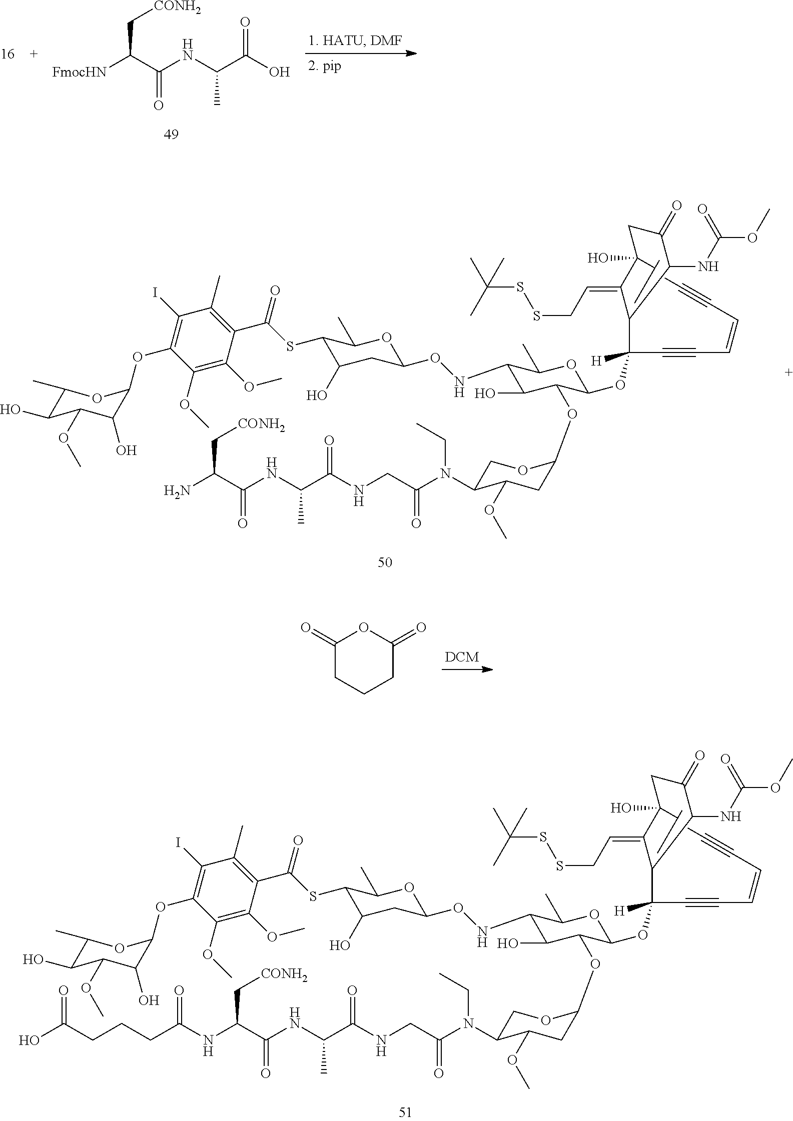

### Preparation of Compound 50:

To compound 16 (14 mg, 10 μmol) in 2 mL of DMF was added compound 49 (8 mg, 19 μmol), 2-(1H-7-azabenzotriazol-1-y1)-1,1,3,3-tetramethyl uranium hexafluorophosphate (HATU, 7 mg, 19 μmol), and 7μL of DIEA. The mixture was stirred for 1 h, then 50 μL of piperidine was added. After 10 min the mixture was purified by HPLC to give compound 50 (12 mg). MS m/z 1620.5 (M+H).

### Preparation of Compound 51:

To compound 50 (12 mg, 8 μmol) in 2 mL of DCM was added glutaric anhydride (1 mg, 9 μmol), and 2 μL of DIEA. The mixture was stirred for 20 min, then evaporated and purified by HPLC to give compound 51 (10 mg). MS m/z 1734.5 (M+H).

### Preparation of Compound 53:

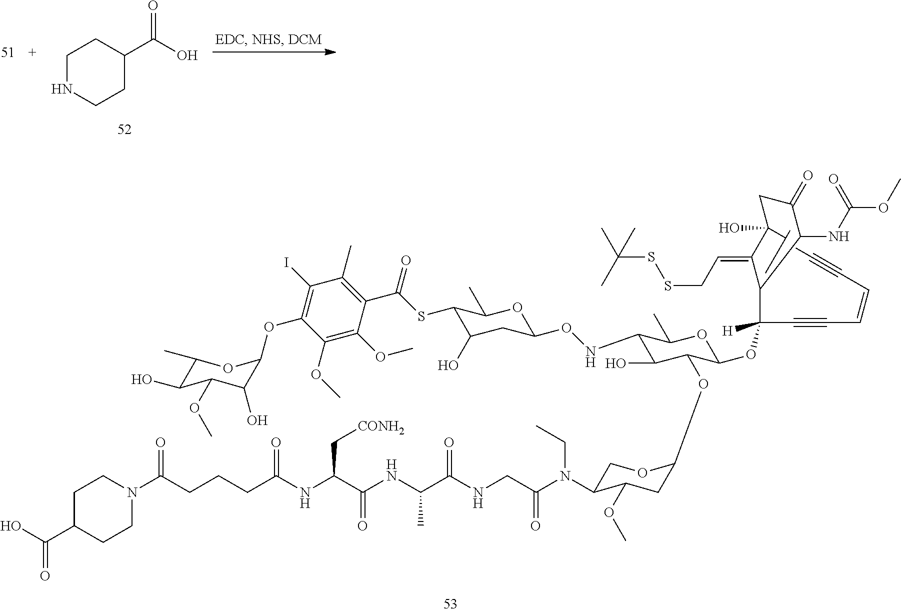

To compound 51 (14 mg, 8.1 μmol) in 2 mL of DCM was added N-hydroxysuccinimide (NHS, 3 mg, 26 μmol), 1-Ethyl-3-(3-dimethylaminopropyl) carbodiimide (EDC, 50 mg, 260 μmol) and the mixture was stirred for 20 min. Then the mixture was washed with water (2 mL), evaporated and the residue was dissolved in 1.5 mL of acetonitrile, 0.5 mL of sat. NaHCO₃ aq. Then compound 52 (10 mg, 80 μmol) was added and the mixture was stirred for 30 min, then the mixture was purified by HPLC to give compound 53 (13 mg). MS m/z 1845.6 (M+H).

### Preparation of Compound 56:

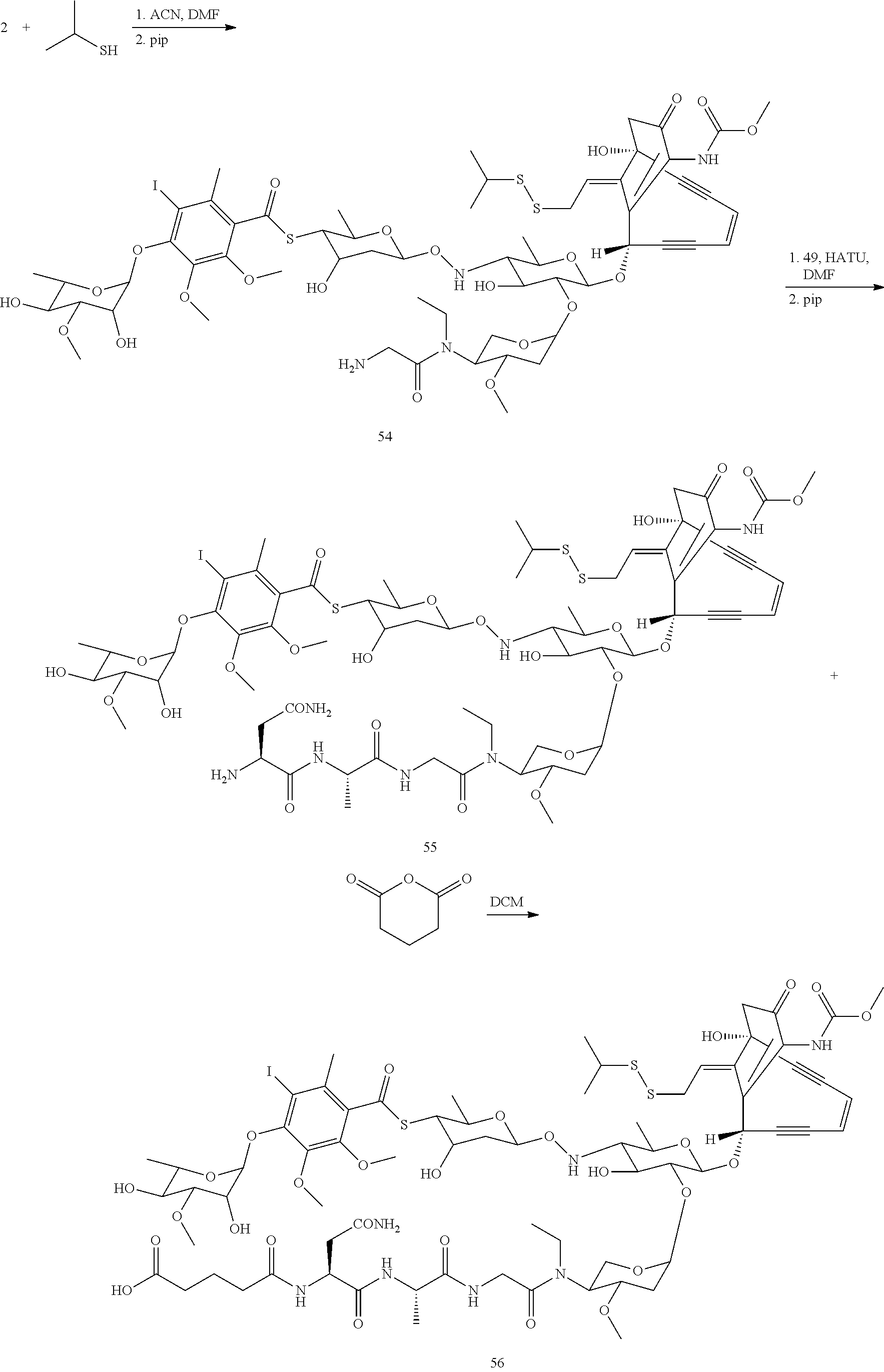

### Preparation of Compound 54:

To compound 2 (100 mg, 0.06 mmol) in 4 mL of acetonitrile and 0.3 mL of DMF was added isopropyl thiol (46 mg, 0.6 mmol). The mixture was stirred for 16 h, then 120 μL of piperidine was added. After 30 min the mixture was purified by HPLC to give compound 54 (50 mg). MS m/z 1421.4 (M+H).

### Preparation of Compound 55:

To compound 54 (15 mg, 10 μmol) in 2 mL of DMF was added compound 49 (8 mg, 19 μmol), HATU (7 mg, 19 umol), and 7 μL of DIEA. The mixture was stirred for 1 h, then 50 μL of piperidine was added. After 10 min the mixture was purified by HPLC to give compound 55 (9 mg). MS m/z 1606.5 (M+H).

### Preparation of Compound 56:

To compound 55 (9 mg, 5.6 μmol) in 2 mL of DCM was added glutaric anhydride (1 mg, 9 μmol), and 2 μL of DIEA. The mixture was stirred for 20 min, then evaporated and purified by HPLC to give compound 56 (8 mg). MS m/z 1720.5 (M+H).

### Preparation of Compound 60:

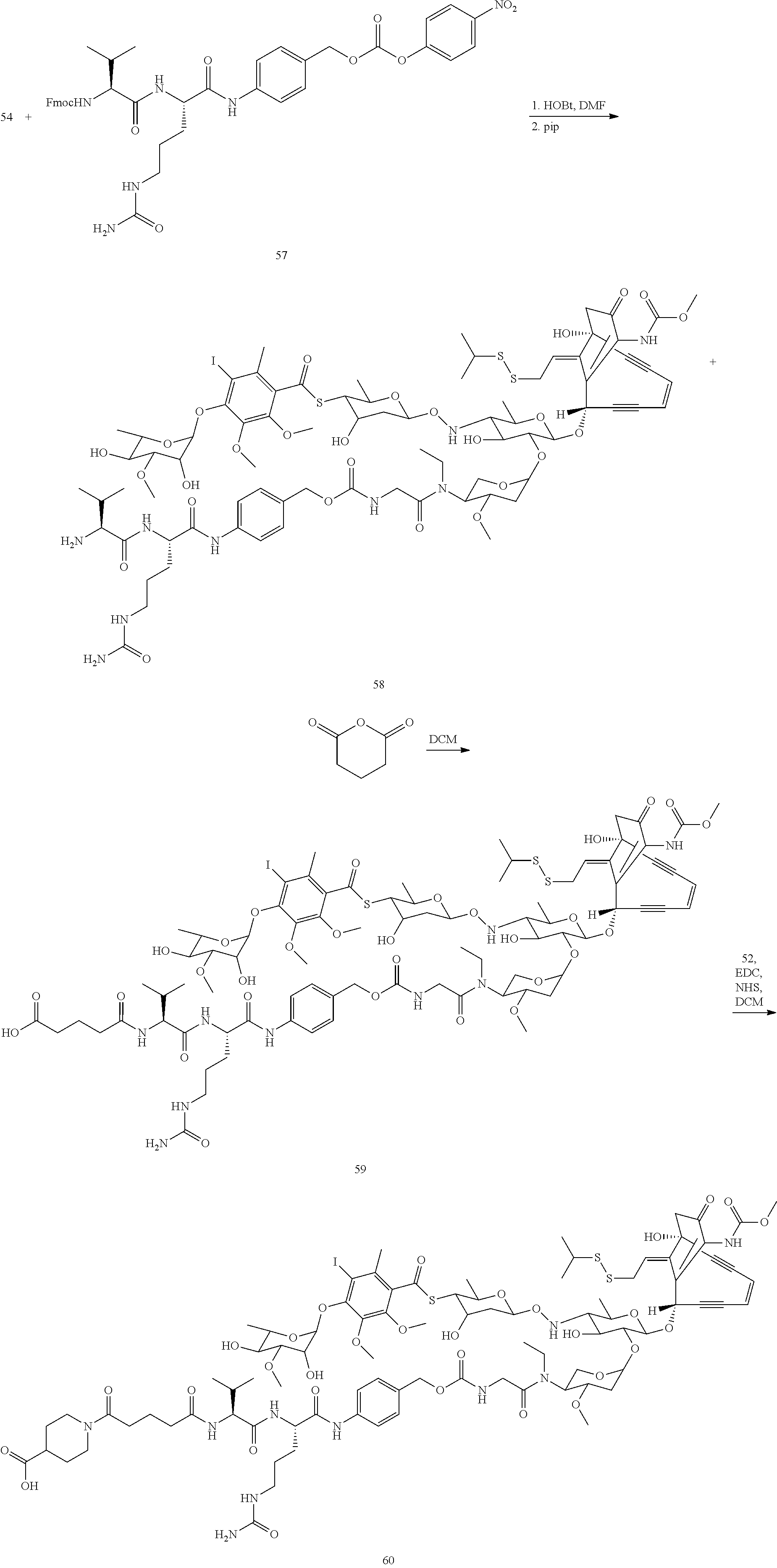

### Preparation of Compound 58:

To compound 54 (23mg, 16 μmol) in 2 mL of DMF was added compound 57 (15 mg, 20 μmol), HOBt (2.5 mg), and 10 μL of DIEA. The mixture was stirred for 1 h, then 40 μL of piperidine was added. After 10 min the mixture was purified by HPLC to give compound 58 (21 mg). MS m/z 1826.5 (M+H).

### Preparation of Compound 59:

To compound 58 (21 mg, 12 μmol) in 2 mL of DCM was added glutaric anhydride (2 mg, 18 μmol), and 3 μL of DIEA. The mixture was stirred for 20 min, then evaporated and purified by HPLC to give compound 59 (18 mg). MS m/z 1940.5 (M+H).

### Preparation of Compound 60:

To compound 59 (16 mg, 8 umol) in 2 mL of DCM was added NHS (3 mg, 26 μmol), EDC (50 mg, 260 μmol) and the mixture was stirred for 20 min. Then the mixture was washed with water (2 mL), evaporated and the residue was dissolved in 2 mL of acetonitrile, 0.5 mL of sat. NaHCO₃ aq. Then compound 52 (10 mg, 80 μmol) was added and the mixture was stirred for 30 min, then the mixture was purified by HPLC to give compound 60 (13 mg). MS m/z 2051.6 (M+H).

### Preparation of Compound 64:

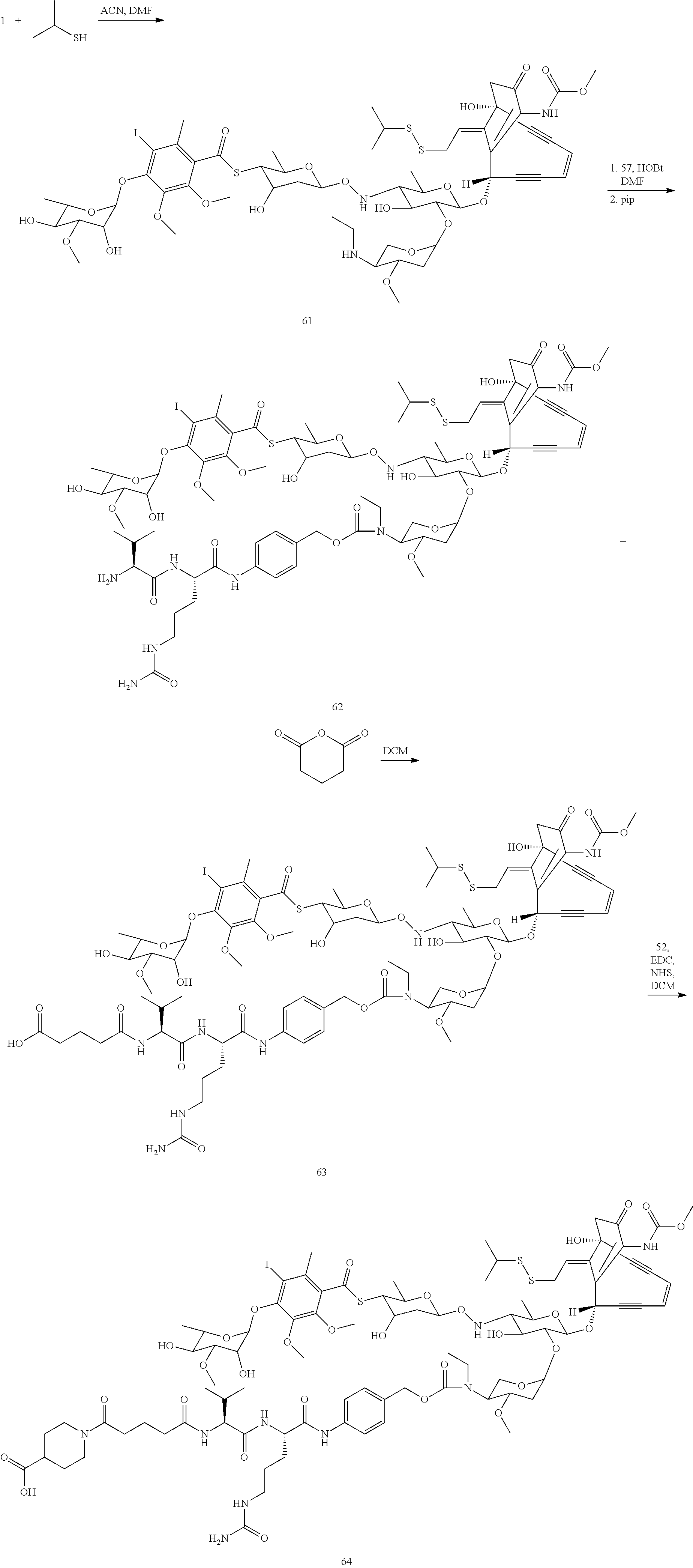

### Preparation of Compound 61:

To compound 1 (86 mg, 0.063 mmol) in 4 mL of acetonitrile and 0.3 mL of DMF was added isopropyl thiol (46 mg, 0.6 mmol). The mixture was stirred for 16 h, then the mixture was purified by HPLC to give compound 61 (25 mg). MS m/z 1364.3 (M+H).

### Preparation of Compound 62:

To compound 61 (22mg, 16 μmol) in 2 mL of DMF was added compound 57 (15 mg, 20 μmol), HOBt (2.5 mg), and 10 μL of DIEA. The mixture was stirred for 16 h, then 40 μL of piperidine was added. After 10 min the mixture was purified by HPLC to give compound 62 (11 mg). MS m/z 1769.5 (M+H).

### Preparation of Compound 63:

To compound 62 (11 mg, 6 μmol) in 2 mL of DCM was added glutaric anhydride (1 mg, 9 μmol), and 2μL of DIEA. The mixture was stirred for 20 min, then evaporated and purified by HPLC to give compound 63 (9 mg). MS m/z 1883.5 (M+H).

### Preparation of Compound 64:

To compound 63 (9 mg, 4.8 μmol) in 2 mL of DCM was added NHS (3 mg, 26 μmol), EDC (50 mg, 260 μmol) and the mixture was stirred for 20 min Then the mixture was washed with water (2 mL), evaporated and the residue was dissolved in 2 mL of acetonitrile, 0.5 mL of sat. NaHCO₃ aq. Then compound 52 (5 mg, 40 μmol) was added and the mixture was stirred for 30 min, then the mixture was purified by HPLC to give compound 64 (8 mg). MS m/z 1994.6 (M+H).

### Preparation of Compound 68:

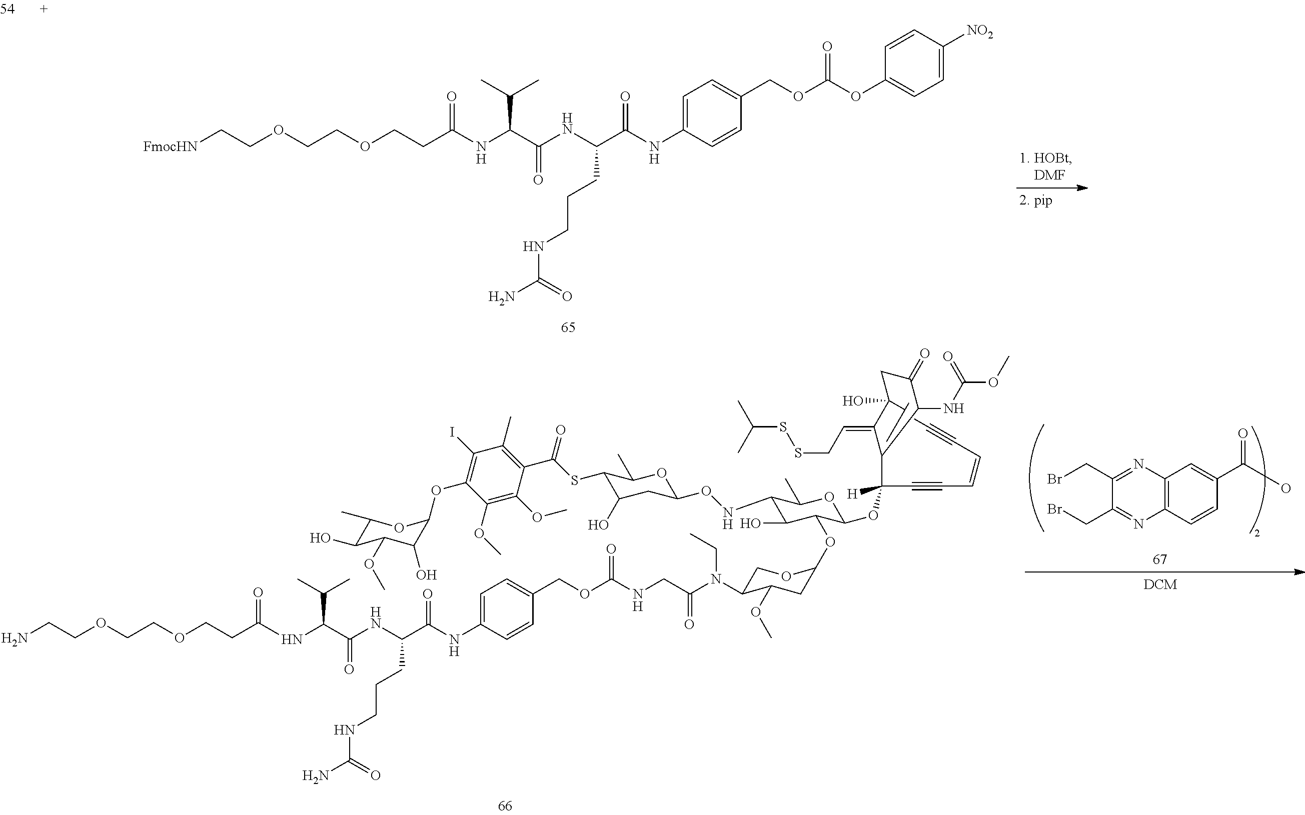

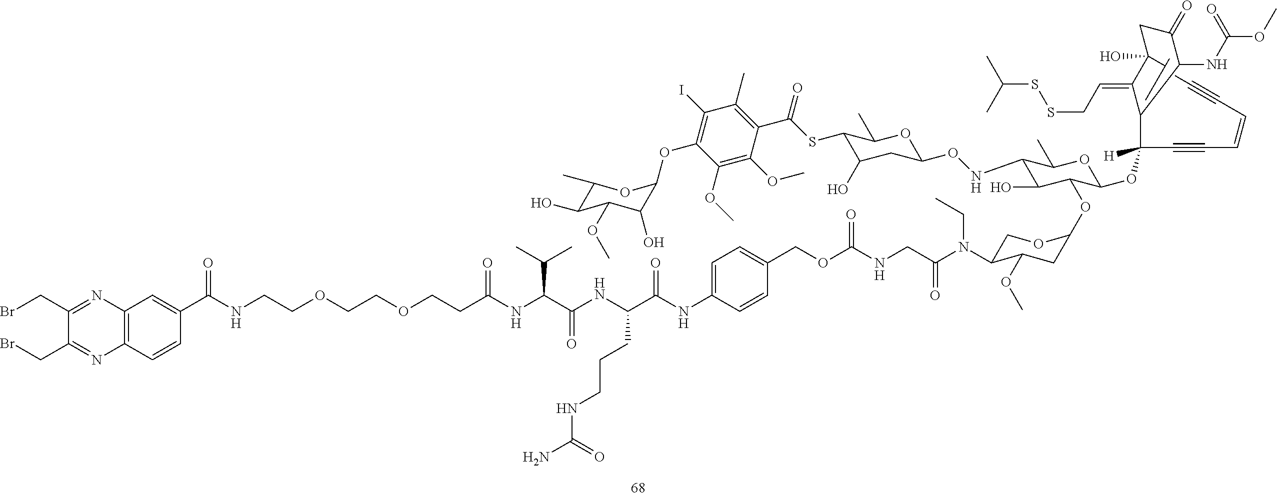

### Preparation of Compound 66:

To compound 54 (10 mg, 7 μmol) in 1 mL of DMF was added compound 65 (8 mg, 8.4 μmol), HOBt (1 mg), and 5μL of DIEA. The mixture was stirred for 2 h, then 40 μL of piperidine was added. After 10 min the mixture was purified by HPLC to give compound 66 (8.6 mg). MS m/z 1985.5 (M+H).

### Preparation of Compound 68:

To compound 66 (8.6 mg, 4.3 μmol) in 2 mL of DCM was added compound 67 (2.1 mg, 3 μmol), and 2 μL of DIEA. The mixture was stirred for 10 min, then evaporated and purified by HPLC to give compound 68 (5 mg). MS m/z 2325.5 (M+H).

### Preparation of Compound 70:

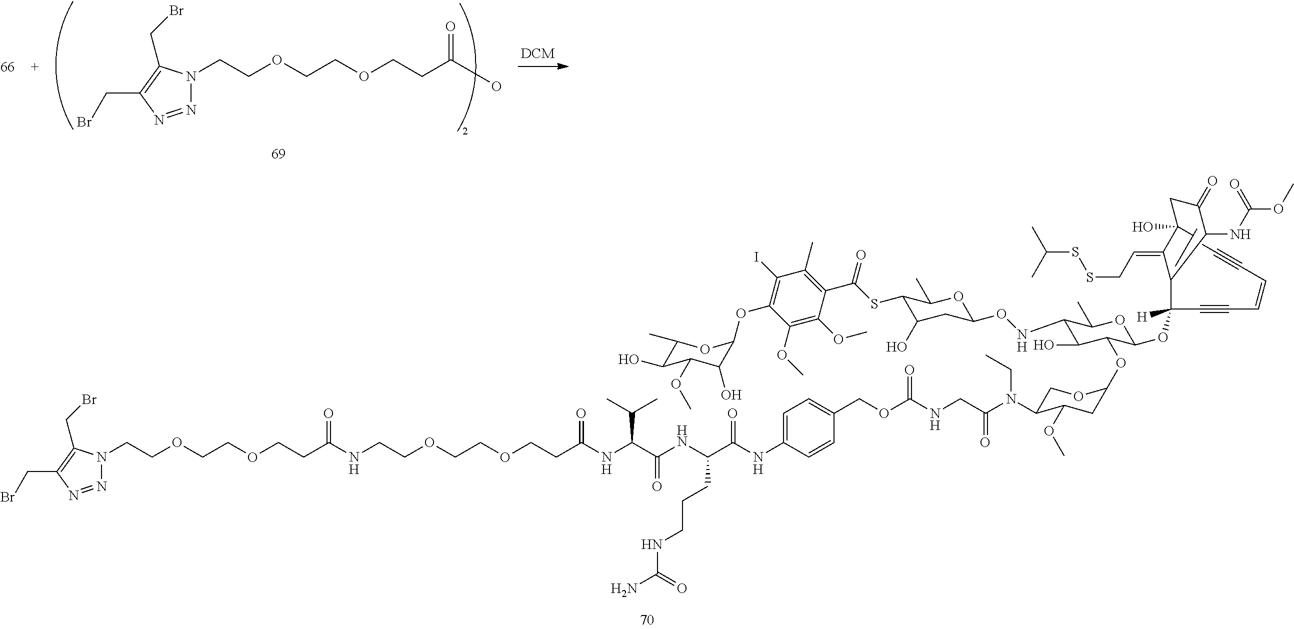

To compound 66 (4.5 mg, 2.3 μmol) in 2 mL of DCM was added compound 69 (3.3 mg, 5 μmol), and 2μL of DIEA. The mixture was stirred for 10 min, then evaporated and purified by HPLC to give compound 70 (4.5 mg). MS m/z 2380.6 (M+H).

### Preparation of Compound 72:

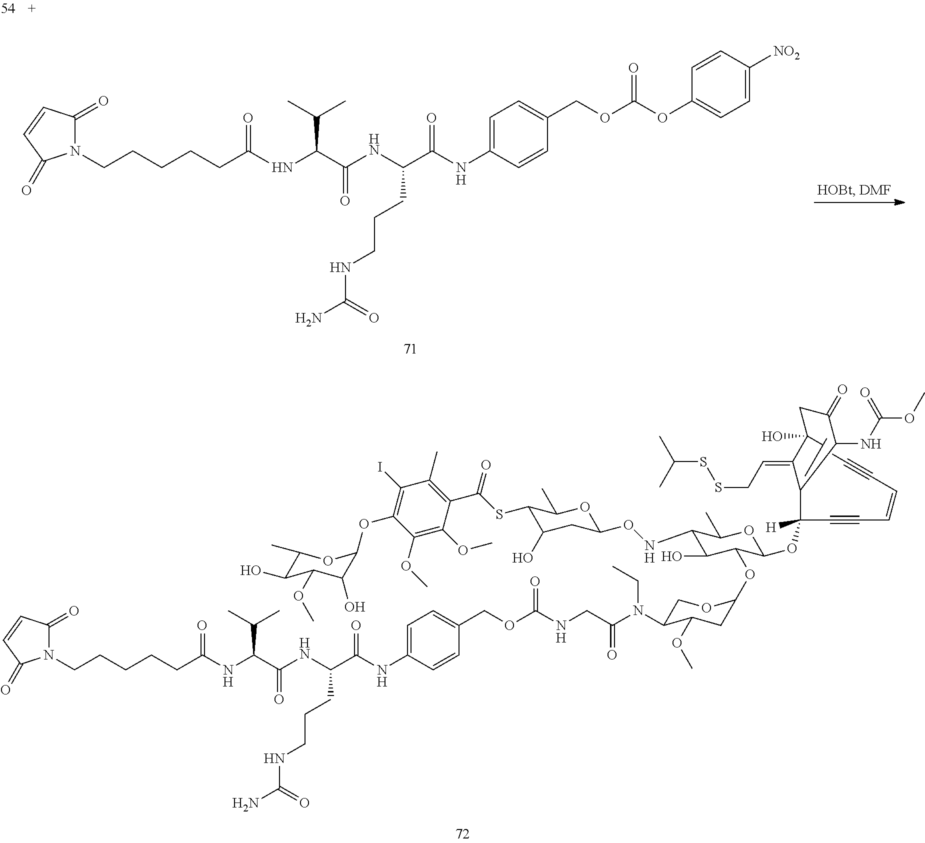

To compound 54 (10 mg, 7 μmol) in 1 mL of DMF was added compound 71 (6 mg, 8.4 μmol), HOBt (1 mg), and 5μL of DIEA. The mixture was stirred for 2 h, then purified by HPLC to give compound 72 (8.6 mg). MS m/z 2019.6 (M+H).

### Preparation of Compound 75:

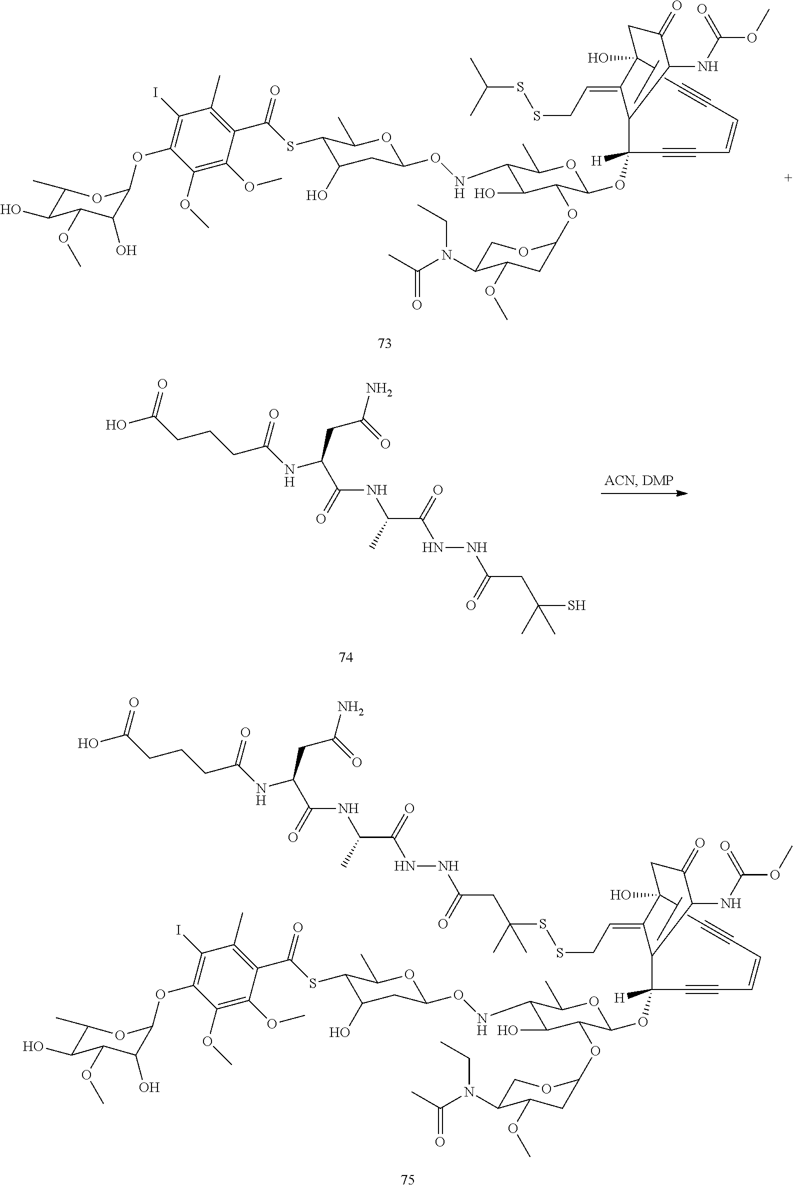

To compound 73 (50 mg, 35 μmol) in 2 mL of DMF and 1 mL of acetonitrile was added compound 74 (32 mg, 71 μmol). The mixture was stirred for 16 h, then purified by HPLC to give compound 75 (25 mg). MS m/z 1777.5 (M+H).

### Preparation of Compound 78:

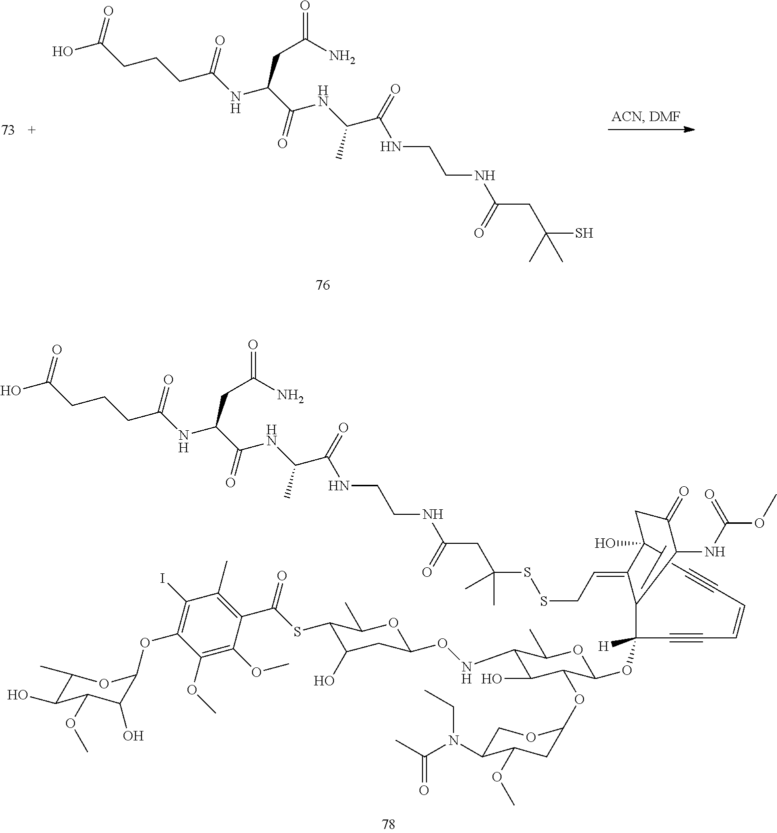

To compound 73 (50 mg, 35 μmol) in 2 mL of DMF and 1 mL of acetonitrile was added compound 76 (34 mg, 71 μmol). The mixture was stirred for 16 h, then purified by HPLC to give compound 78 (23 mg). MS m/z 1805.5 (M+H).

### Preparation of Compound 81 and 81a:

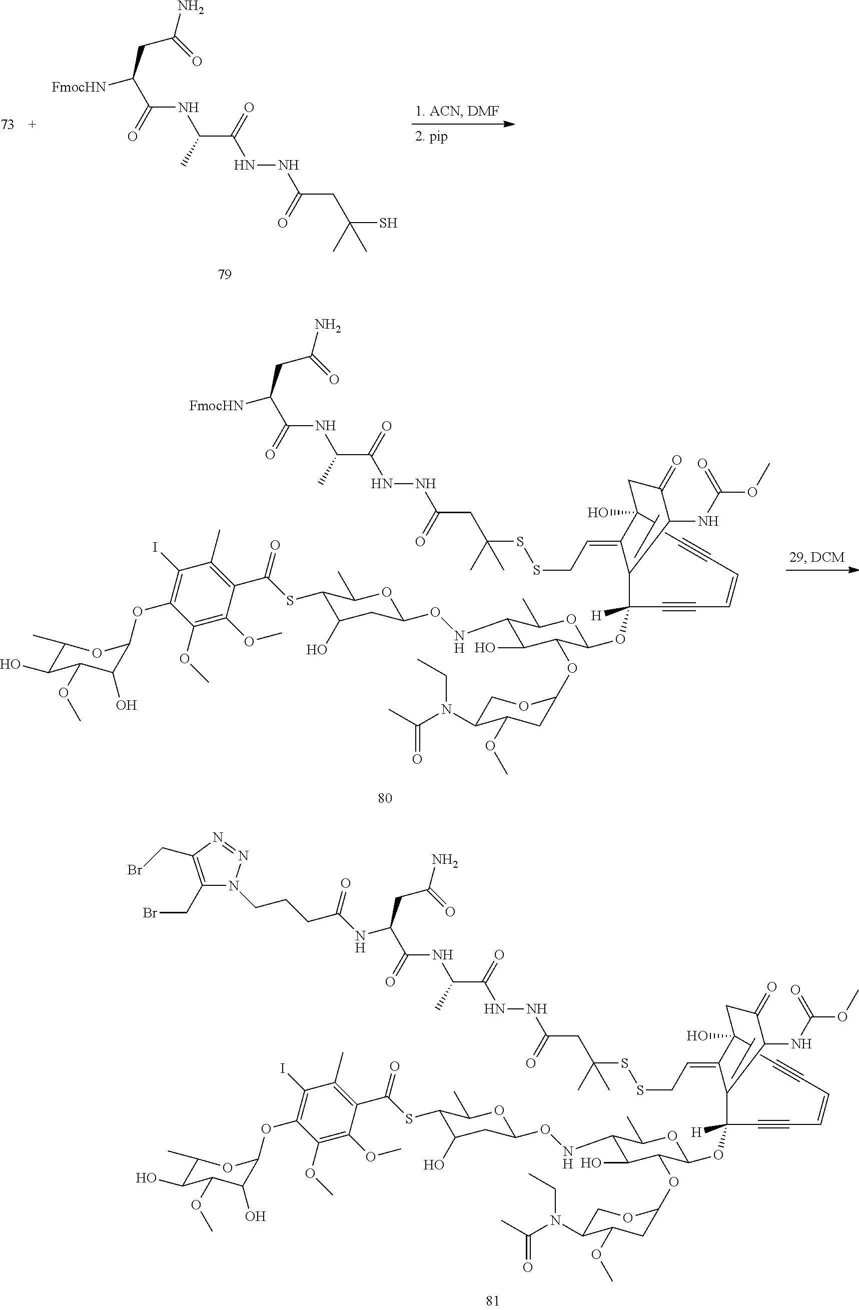

### Preparation of Compound 80:

To compound 73 (50 mg, 35 μmol) in 2 mL of DMF and 1 mL of acetonitrile was added compound 79 (39 mg, 71 μmol). The mixture was stirred for 1 h, then 60 μL of piperidine was added. After 10 min the mixture was purified by HPLC to give compound 80 (18 mg). MS m/z 1663.5 (M+H).

### Preparation of Compound 81:

To compound 80 (18 mg, 11 μmol) in 2 mL of DCM was added compound 29 (8.6 mg, 13 μmol), and 3 μL of DIEA. The mixture was stirred for 20 min, then evaporated and purified by HPLC to give compound 81 (12 mg). MS m/z 1984.4 (M+H).

### Preparation of Compound 84 and 84a:

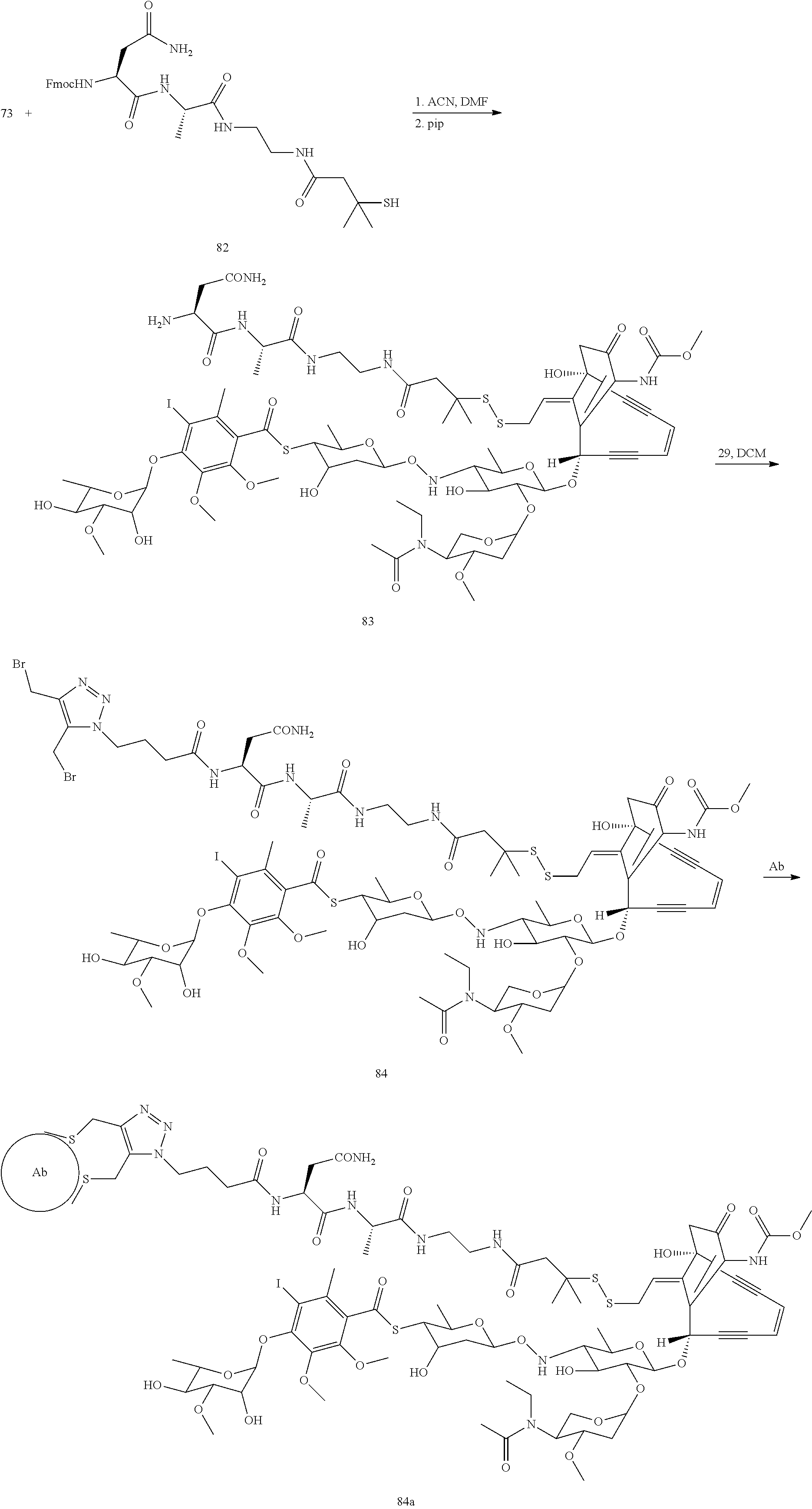

### Preparation of Compound 83:

To compound 73 (50 mg, 35 μmol) in 2 mL of DMF and 1 mL of acetonitrile was added compound 82 (41 mg, 71 μmol). The mixture was stirred for 1 h, then 60 μL of piperidine was added. After 10 min the mixture was purified by HPLC to give compound 83 (20 mg). MS m/z 1691.5 (M+H).

### Preparation of Compound 84:

To compound 80 (19 mg, 11 μmol) in 2 mL of DCM was added compound 29 (8.6 mg, 13 μmol), and 3 μL of DIEA. The mixture was stirred for 20 min, then evaporated and purified by HPLC to give compound 84 (13 mg). MS m/z 2012.4 (M+H).

Preparation of cMet-ADC (Compounds 8a and 81a)

Anti-cMet antibody was reduced by TCEP (tris(2-carboxyethyl)phosphine), up to 20 mM. The excess of TCEP was removed by gel-filtration chromatography or centrifugal filtration. Added organic solvent (up to 50%) to antibody solution. Compound 8 or 81 was dissolved in Acetonitrile/water solution and added to the reduced antibody with compound 8 or 81/antibody ratio from 3.5 to 6. After few hours' incubation at room temperature, unconjugated compound 8 or 81 was removed by gel-filtration chromatography or centrifugal filtration. The cMet-ADC was characterized by HPLC. The drug antibody ratio (DAR) was calculated based on UV-VIS or HIC-HPLC.

### EXAMPLE 2

This example provides the results of EC50 assays (nM) of the designated drug conjugated antibodies measured in vitro in specified cells. The antibody used was an anti-HER2 IgG class of antibody. Seven breast cancer cell lines with various level of Her2 expression as indicated with plus or minus signs in the table below were plated in 96 well plate. The ADCs as listed under Drug-Linker ID were serial diluted and added onto cells for treatment for 5 days. At the end of the study, cell proliferation was measured by Promega's CellTitreGlo. EC50 (in nM) was shown below and determined as the concentration of 50% cell growth inhibition. The selection criteria for a successful compound includes high efficacy, such as killing cell lines with high expression of the target receptor, with EC50 less than 2 nM. Also, the successful candidate should have low toxicity and good therapeutic window, as determined by relatively low killing of the control cell line (MDA468) with low expression of the target receptor. Compounds 15, 30, 34, 37, 40, 56, 75, 78, 81, and 84, were selected as successful candidates with high efficacy and good therapeutic window. Compounds 44 and 51 have low toxicity, but failed in efficacy in some cell lines. Compound 72 has high toxicity and low efficacy in several cell lines.

| | | | MDA- | | | | |
|---|---|---|---|---|---|---|---|
| Drug-Linker | SKBR-3 | BT474 | 453 | JIMT-1 | MCF-7 | MDA468 | HCC1954 |
| ID | (Her2+++) | (Her2+++) | (Her2++) | (Her2+++) | (Her2+/−) | (Her2−) | (Her2+++) |
| 15 | 0.17 | 1.46 | | 0.17 | >100 | | 0.064 |
| 30 | 0.065 | 1.41 | 0.05 | 0.09 | 0.5 | 24 | |
| 34 | 0.057 | 0.57 | 0.162 | 0.022 | 0.033 | 5.12 | |
| 37 | 0.234 | 1.53 | 0.33 | 0.141 | 0.33 | 16.9 | |
| 40 | 0.657 | 0.9 | 0.1 | 0.35 | 90 | 41.7 | |
| 44 | 0.223 | 2.49 | 0.08 | >100 | >100 | >100 | |
| 51 | 0.127 | 0.639 | 0.1 | >100 | >100 | >100 | |
| 56 | 0.04 | 0.42 | 0.05 | 0.238 | 22 | 32 | |
| 72 | 0.171 | 22.4 | 5.4 | 0.084 | | 2 | 0.09 |
| 75 | 0.015 | 0.237 | 0.02 | 0.021 | 0.026 | 36.7 | |
| 78 | 0.134 | 0.4 | 0.01 | 0.44 | >100 | >100 | |
| 81 | 0.023 | 0.418 | 0.1 | 0.025 | 0.041 | 12.98 | 0.01 |
| 84 | 0.07 | 0.96 | 0.041 | 0.119 | 12 | 63.8 | |

### EXAMPLE 3

This example provides the results of EC50 assays (nM) of designated ADCs described herein measured in vitro in specified cells. The antibody used targets a receptor tyrosine kinase on cell surface. Eight cancer cell lines with various level of receptor expression as indicated with plus or minus signs in the table below were plated in 96 well plate. The ADCs as listed under Drug-Linker ID were serial diluted and added onto cells for treatment for 5 days. At the end of the study, cell proliferation was measured by Promega's CellTitreGlo. EC50 (in nM) was shown below and determined as the concentration of 50% cell growth inhibition. The selection criteria for a successful compound includes high efficacy, such as killing cell lines with high expression of the target receptor, with EC50 less than 2 nM. Also, the successful candidate should have low toxicity and good therapeutic window, as determined by relatively low killing of the control cell lines (T-47D and H520) with low expression of the target receptor. Compounds 8a, 14a, 30a, 81a, and 84a were selected as successful candidates with high efficacy and good therapeutic window. Compounds 23a and 18a have low toxicity, but did not show efficacy in some cell lines.

| Drug- | H1993 | H292 | HCC827 | Hs746T | SNU-5 | T-47D | A549 | H520 |
|---|---|---|---|---|---|---|---|---|
| Linker ID | (+) | (+) | (+) | (+) | (+) | (−) | (+) | (−) |
| 8a | 0.05 | 1.4 | 0.01 | | 0.121 | 80 | 0.46 | 30 |
| 23a | 0.306 | 3.5 | 40 | 0.21 | 1.23 | 20 | | |
| 14a | 0.231 | 0.5 | | | | | | >100 |
| 18a | 0.265 | >100 | | | | | 10 | 10 |
| 30a | 0.055 | | 0.36 | | | >100 | 0.428 | |
| 81a | 0.002 | 1 | 0.055 | 2 | 0.057 | >100 | 0.015 | 80 |
| 84a | 0.08 | | 0.36 | | | >100 | 0.154 | |

### EXAMPLE 4

This example provides the results for the in vivo efficacy of compounds 8a and 81a on a variety of Human Xenograft Tumour Growth in Nude Mice

### Animals:

Female Nu/Nu mice at 5-7 weeks of age (from Charles River) were used in the studies. Upon receipt, mice were housed 5 mice per cage in a room with a controlled environment. Rodent chow was provided and water ad libitum . Mice were acclimated to laboratory conditions for 72 hours before the start of dosing. Animals' health status was determined during the acclamation period. Each cage was identified by group number and study number, and mice were identified individually by ear tags.

Study Design and Dosing Regimen for Conjugate #81a: refer to Table 1.

TABLE 1

| Tumor | | Animals per | | |
|---|---|---|---|---|
| model | Groups | Group | Treatment volume/route | Dose/Frequency |
| HCC827 | 1 | 7 | PBS/0.2 ml, IV | 0 mg/kg/once |
| | 2 | 7 | Compound 81a/0.2 ml. IV | 3 mg/kg/once |
| H292 | 1 | 7 | PBS/0.2 ml, IV | 0 mg/kg/once |
| | 2 | 7 | Compound 81a/0.2 ml. IV | 3 mg/kg/once |
| H292 | 1 | 9 | PBS/0.2 ml, IV | 0 mg/kg/Q1W × 3 |
| | 2 | 10 | Compound 81a/0.2 ml. IV | 3 mg/kg/Q1W × 3 |
| U87MG | 1 | 10 | PBS/0.2 ml, IV | 0 mg/kg/Q1W × 3 |
| | 2 | 10 | Compound 81a/0.2 ml. IV | 3 mg/kg/Q1W × 3 |
| H1993 | 1 | 10 | PBS/0.2 ml, IV | 0 mg/kg/Q1W × 3 |
| | 2 | 10 | Compound 81a/0.2 ml. IV | 3 mg/kg/Q1W × 3 |

Study Design and Dosing Regimen for Conjugate #8a : refer to Table 2.

TABLE 2

| | | Animals | | |
|---|---|---|---|---|
| Tumor | | per | | Dose/ |
| model | Groups | Group | Treatment volume/route | Frequency |
| HCC827 | 1 | 7-10 | PBS/0.2 ml, IV | 0 mg/kg once |
| | 3 | 7-10 | Compound 8a/0.2 ml. IV | 3 mg/kg once |
| H292 | 1 | 7-10 | PBS/0.2 ml, IV | 0 mg/kg once |
| | 3 | 7-10 | Compound 8a/0.2 ml. IV | 3 mg/kg once |
| U87MG | 1 | 7-10 | PBS/0.2 ml, IV | 0 mg/kg once |
| | 2 | 7-10 | Compound 8a/0.2 ml. IV | 3 mg/kg once |
| H1975 | 1 | 7-10 | PBS/0.2 ml, IV | 0 mg/kg once |
| | 3 | 7-10 | Compound 8a/0.2 ml. IV | 3 mg/kg once |

### Procedures

### 1. Tumor Cell Inoculation and Establishments of Tumors:

a. U87MG, H292, H1993, H1975 and HCC827 cell lines were obtained from ATCC (Manassas, Va.). Human NSCLC cell lines HCC827, H292 H1993 and H1975 were cultured and expanded with 10% FBS RPMI medium, U87MG with DMEM medium at 37° C. in a 5% carbon dioxide humidified environment. The cells were cultured, and passage as needed for a period of 2 weeks and then harvested with 0.25% trypsin (Corning 25-050-CI). 7 million cells of HCC827, 5 million cells of H292, H1993 and U87MG in a total of 0.2 ml 1:1 ratio of mixture of HBSS (Hank's balanced salt solution) and matrigel (Corning 354234) were injected subcutaneously into the upper right flank of each mouse respectively. All mice were ear tagged for identification.
b. Tumor growth was monitored by tumor volume measurement using a digital caliper starting day 5-7 after inoculation, and followed 2 times per week until tumor volume reaches˜150-250 mm³.

### 2. Treatments:

a. Once tumors were staged to the desired volume, animals were randomized and mice with very large or small tumors were culled. Mice were divided into treatment groups with animal numbers per group as indicated in the study design (Table 1 and Table 2) for each tumor model.
b. Mice were then treated with either vehicle (PBS, 0.2 ml IV) or ADC Compound 81a or Compound 8 a according to the study design.

### 3. Tumor Volume, Body Weight Measurement and Study End Points

a. Tumor volumes were measured by using a digital caliper twice weekly through the whole experiment period. The volume was calculated using the formula: Volume (mm³)=[Length (mm)×Width (mm)²]/2. TGI (tumor growth inhibition %) was calculated using the formula: TGI=[(Last Volume Measurement of vehicle Group—Volume of Treatment group at the same last day of vehicle control)/(Last Volume Measurement of Vehicle Group]×100.
b. Body weight of each mouse was weighed twice weekly by an electric balance.
c. Tumor growth responses were monitored until tumor load reached IACUC protocol limits (2000 mm³) or when animal body weight loss reaches 20%.

### Data Analysis

Raw data of tumor volume and body weight were recorded in an Excel file with Microsoft Office. Tumor volume and body weight graphs were generated by GraphPad Prism 6.0. Data statistical analysis was done by unpaired t-test or one-way ANOVA compared to PBS treated control group.

### Results

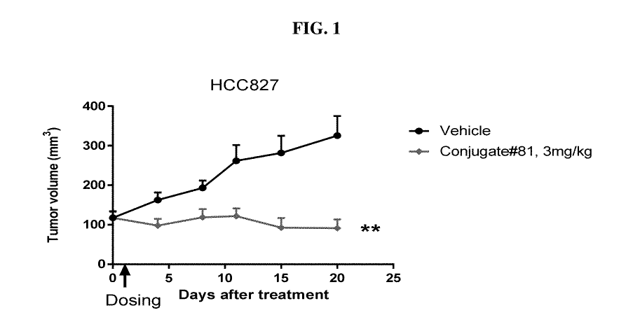

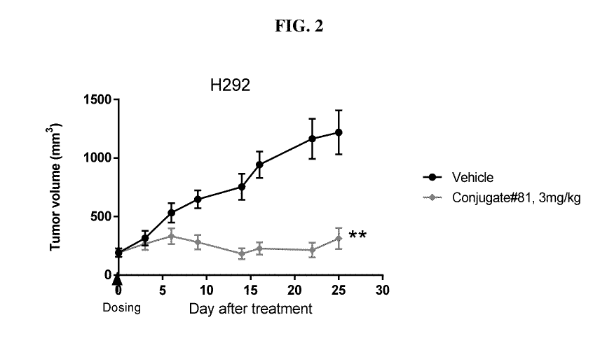

As shown by FIGS. 1 and 2, compound 81a significantly inhibited the growth of HCC827 and H292 tumor in nude mice at 3 mg/kg IV, single dose. Bars represent group averages±SEM, N=7/group. ** P<0.01 vs. Vehicle (unpaired t test).

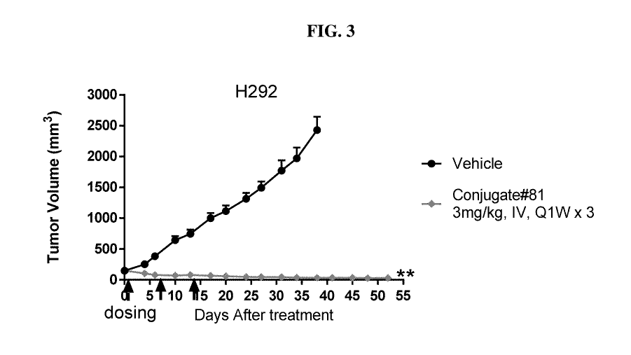

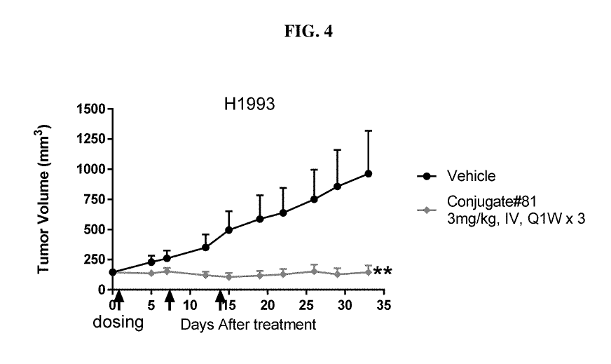

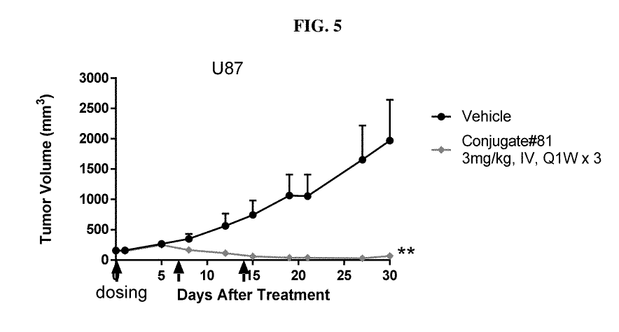

FIGS. 3, 4, and 5 show that compound 81a significantly inhibited the tumor growth of H292, H1993, and U87MG tumor in nude mice at 3 mg/kg IV, once weekly for three treatment. Bars represent group averages±SEM, N=9/group. ** P<0.01 vs. Vehicle (unpaired t test).

Tumor growth inhibition (TGI) is show in Table 3 below.

TABLE 3

| Tumor model | Treatment | TGI % |
|---|---|---|
| H292 | Compound 81a, 3 mg/kg, IV single | 75.30 |
| | dose | |
| H292 | Compound 81a, 3 mg/kg, IV. Q1W × 3 | 98.64% |
| H1993 | Compound 81a, 3 mg/kg, IV. Q1W × 3 | 85.04% |
| HCC827 | Compound 81a, 3 mg/kg, IV. single | 71.91% |
| | dose | |
| U87MG | Compound 81a, 3 mg/kg, IV | 96.62% |
| | Q1W × 3 | |

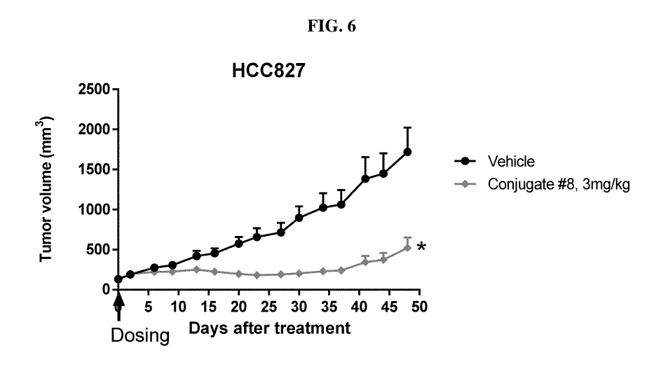

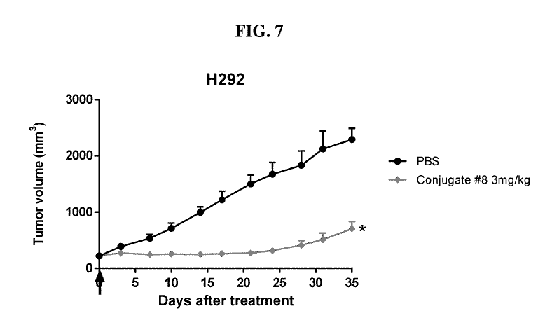

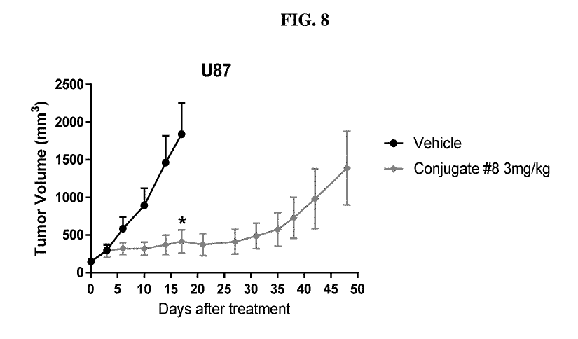

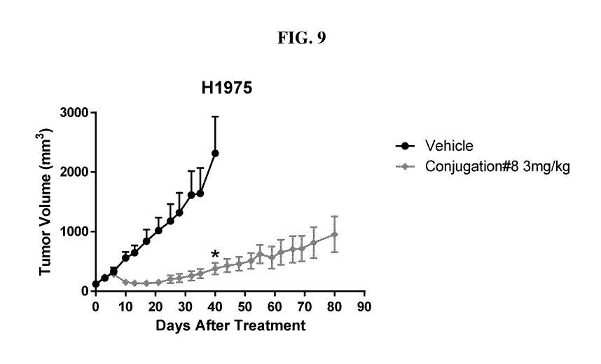

As shown by FIGS. 6, 7, 8, and 9 compound 8a significantly inhibited the growth of HCC827, H292, U87, and H1975 tumor in nude mice at 3 mg/kg IV, single dose. Bars represent group averages±SEM, N=10/group. ** P<0.05 vs. Vehicle (one way ANOVA, Dunnett's multiple comparison test).

Tumor growth inhibition (TGI) is show in Table 4 below.

TABLE 4

| Tumor model | Treatment | TGI % |
|---|---|---|
| H292 | Compound 8a, 3 mg/kg, IV single | 69.49% |
| | dose | (Day38) |
| HCC827 | Compound 8a, 3 mg/kg, IV. single | 69.5% |
| | dose | (Day48) |
| U87MG | Compound 8a, 3 mg/kg, IV. single | 77.40% |
| | dose | (Day17) |
| H1975 | Compound 8a, 3 mg/kg, IV. Single | 83.58% |
| | dose | (Day40) |

We claim:

1. An ADC comprising a structure of Formula II
AbL¹-L²-D)ₙ (II)
or a pharmaceutically acceptable salt thereof, wherein:
Ab is a monoclonal antibody;
L¹-L²together are a linker selected from the group consisting of: 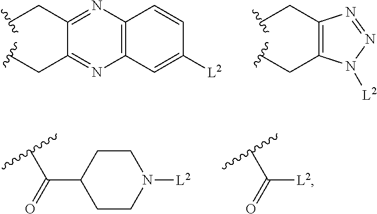
wherein the wavy line indicates a point of attachment to an Ab;
L² is a linker; wherein L² is selected from the group consisting of an amino acid, a peptide, —(CH₂)ₘ—, —(CH₂CH₂O)ₘ—, PAB, Val-Cit-PAB, Val-Ala-PAB, Ala-Ala-Asn-PAB, and combinations thereof, wherein m is an integer from 0 to 10;
D is calicheamicin; and
n is 1, 2, 3, 4, 5, 6, 7, 8, 9, or 10.

2. The ADC of claim 1, wherein D has the structure of Formula III 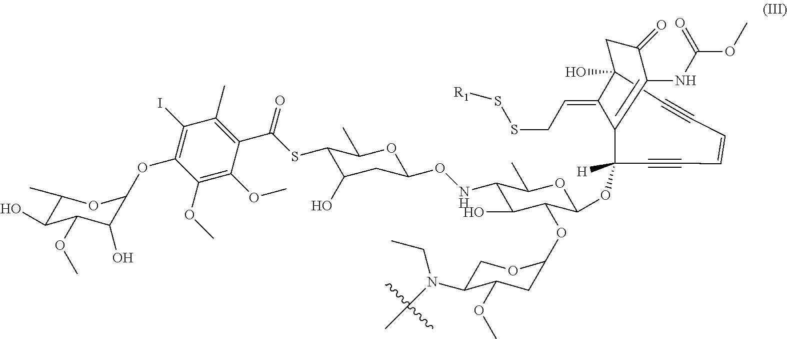
or a pharmaceutically acceptable salt thereof;
wherein the wavy line indicates the point of attachment to L²; and
wherein R1 is selected from the group consisting of C1-C8 alkyl, —(CH₂CH₂O)ₙ—, isopropyl, glucose, galactose, mannose, glucosamine, C1-C8 alkyl-OH, and combinations thereof, and
where n=1-30

3. The ADC of claim 1, or a pharmaceutically acceptable salt thereof, wherein R1 is selected from the group consisting of: isopropyl, 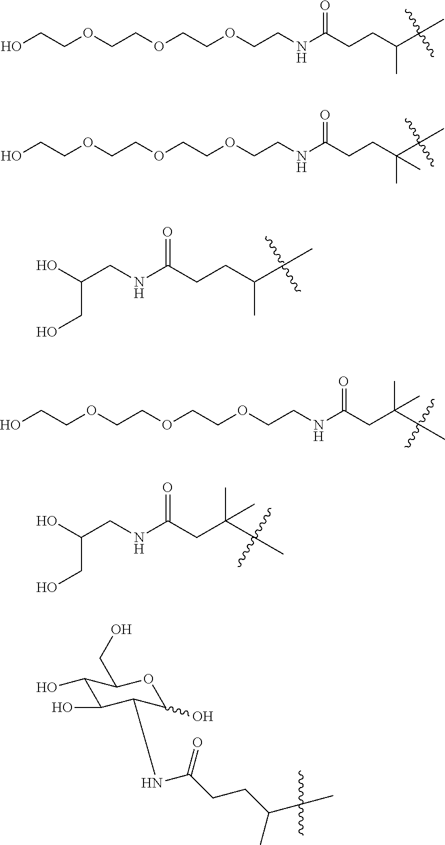 [SMILES: *C(C)CCC(=O)NCCOCCOCCOCCO ; *C(C)(C)CCC(=O)NCCOCCOCCOCCO ; *C(C)CCC(=O)NCC(O)CO ; *C(C)(C)CC(=O)NCCOCCOCCOCCO ; *C(C)(C)CC(=O)NCC(O)CO ; *C(C)CCC(=O)NC1C(O)OC(CO)C(O)C1O]
wherein the wavy line indicates a point of attachment to the sulfur on calicheamicin.

4. The ADC of claim 1, wherein the structure of Formula II has a structure selected from the group consisting of: 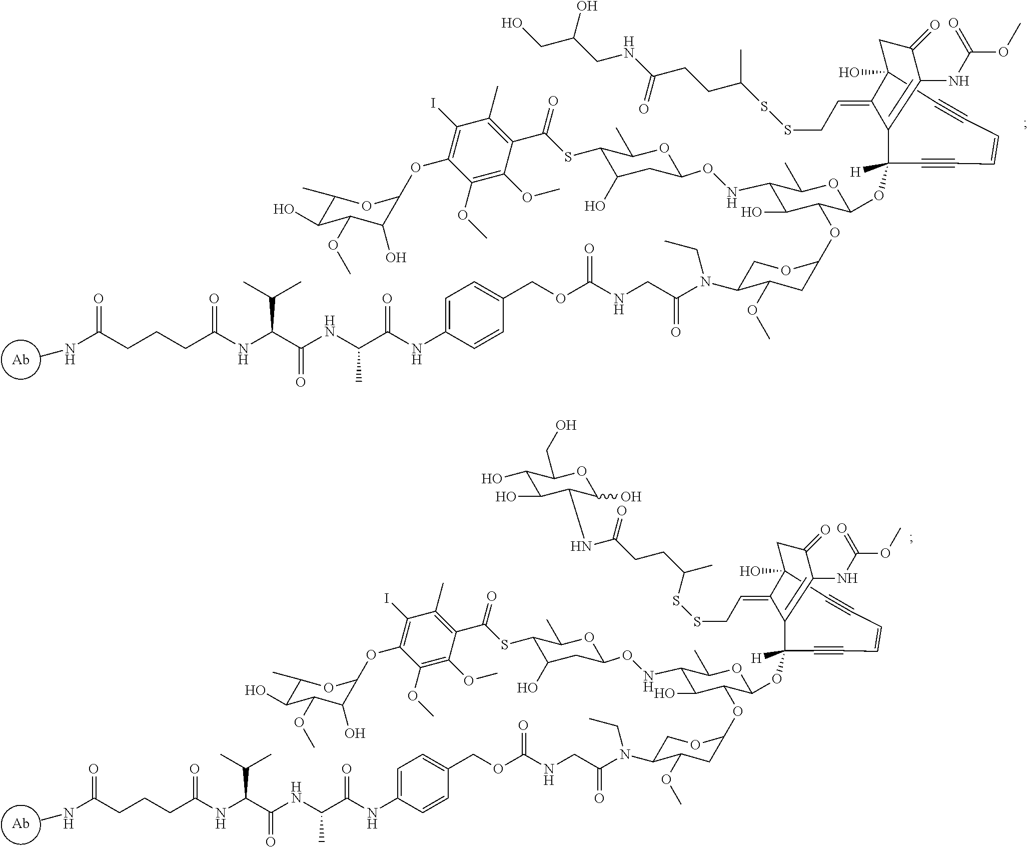 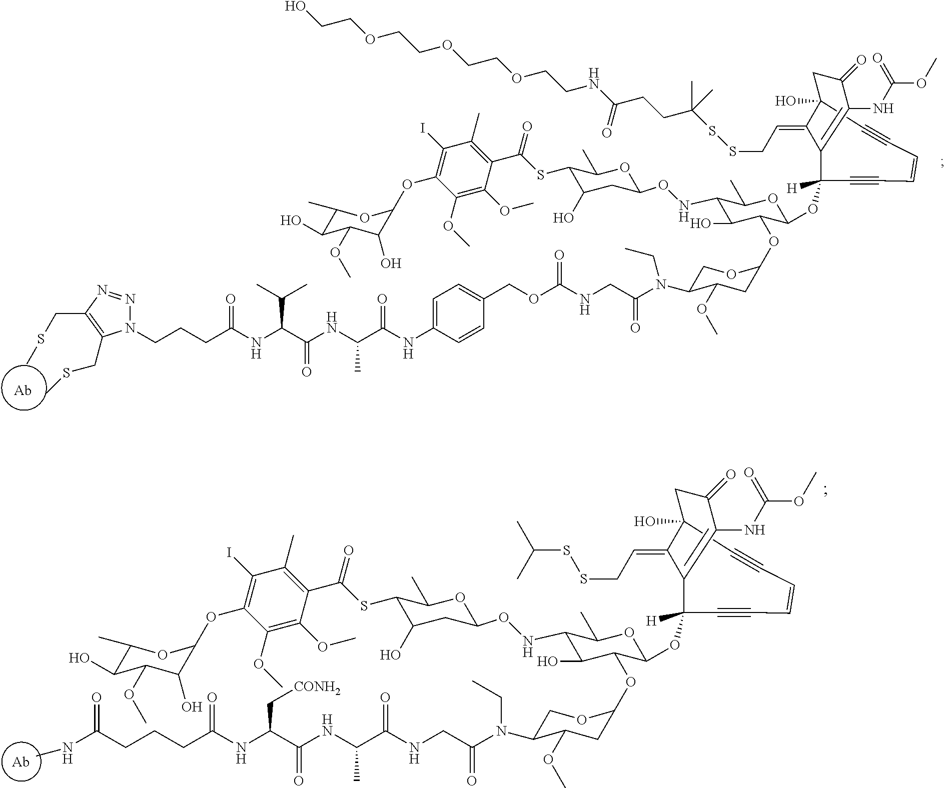 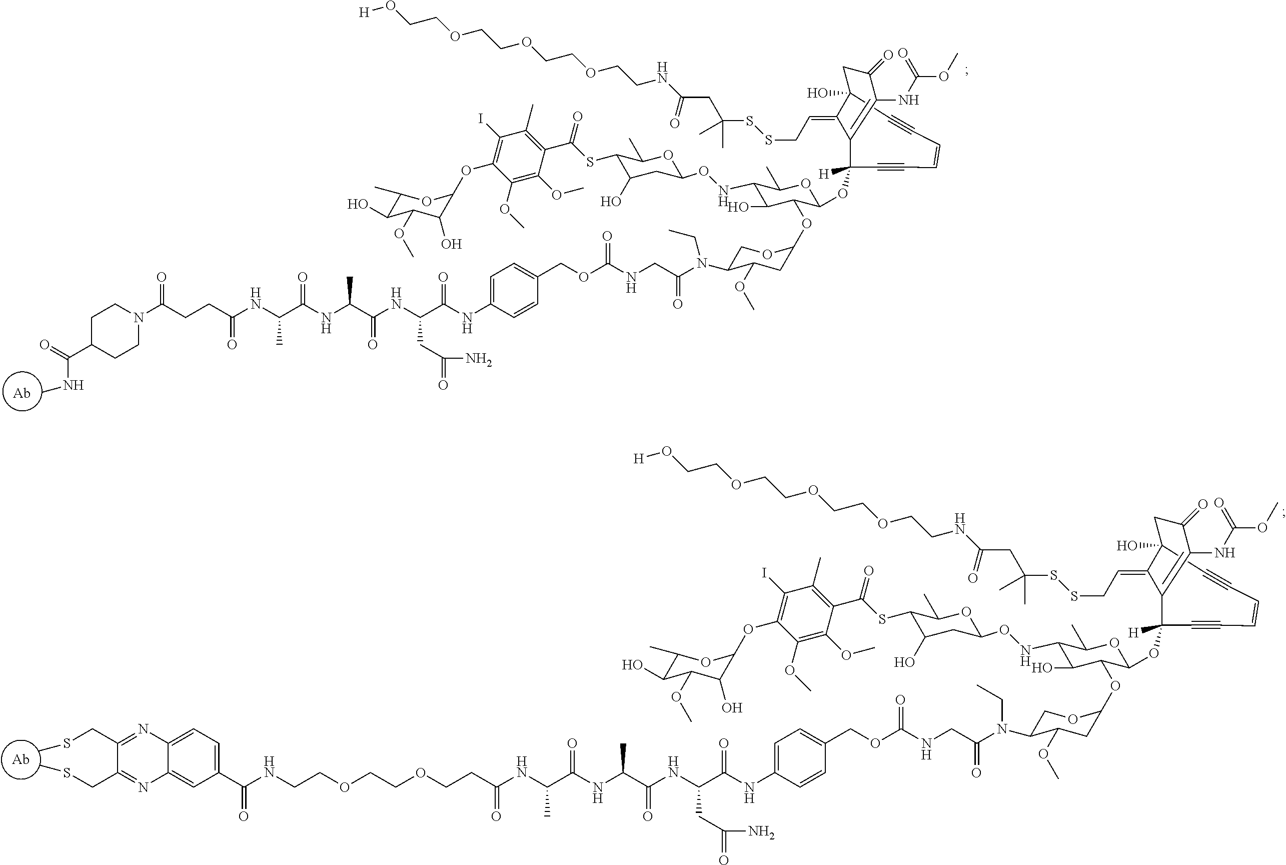 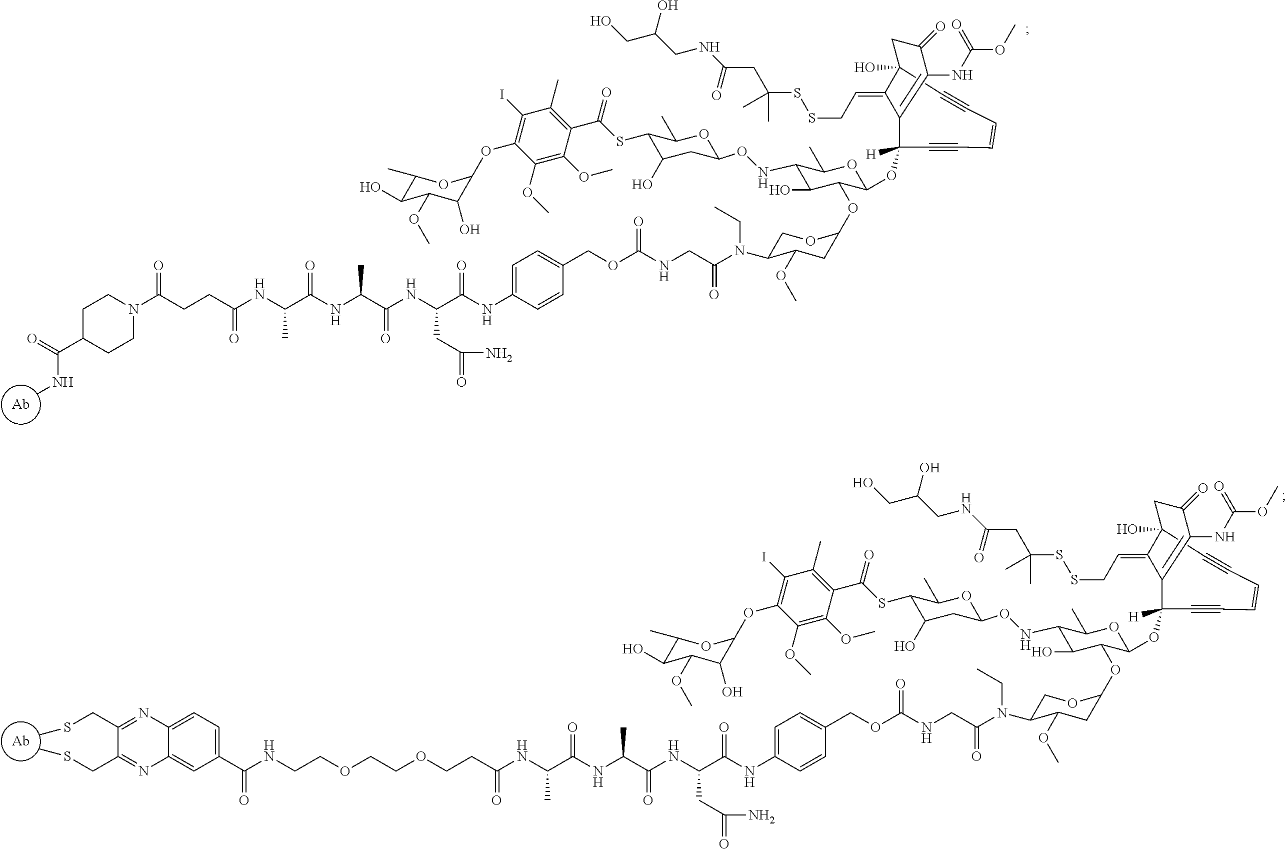 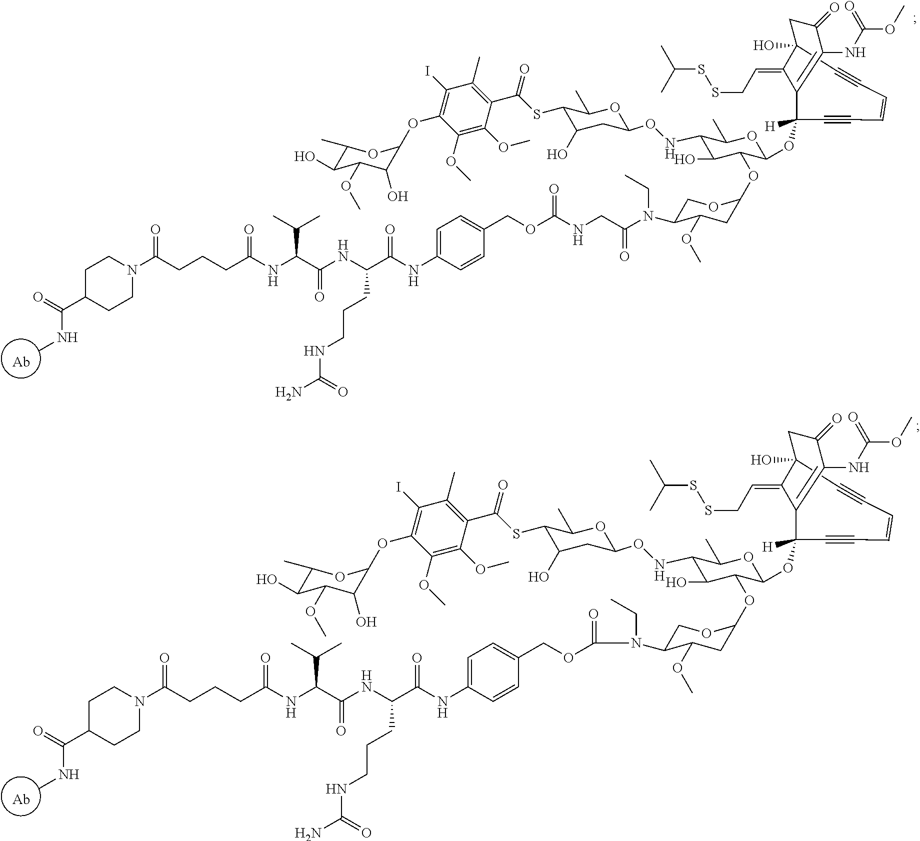 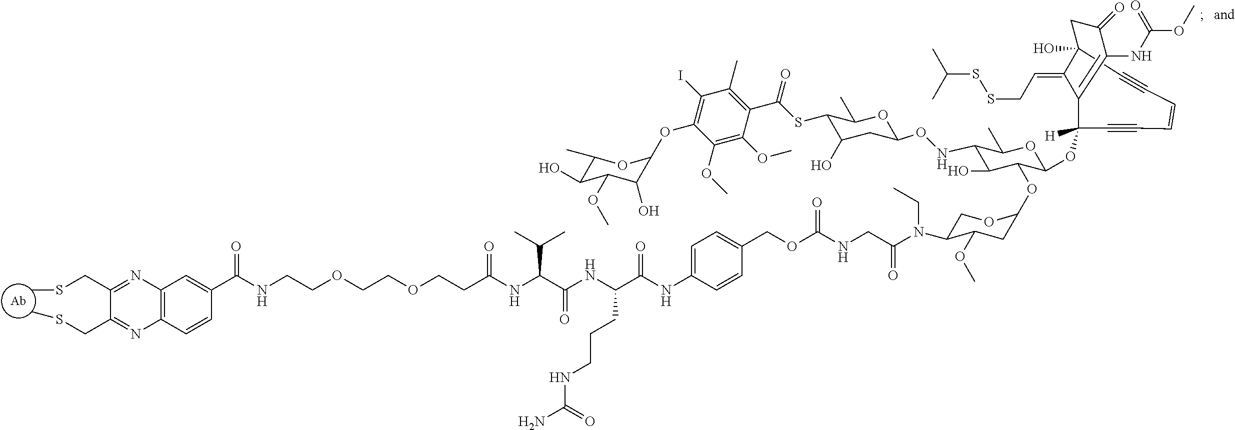 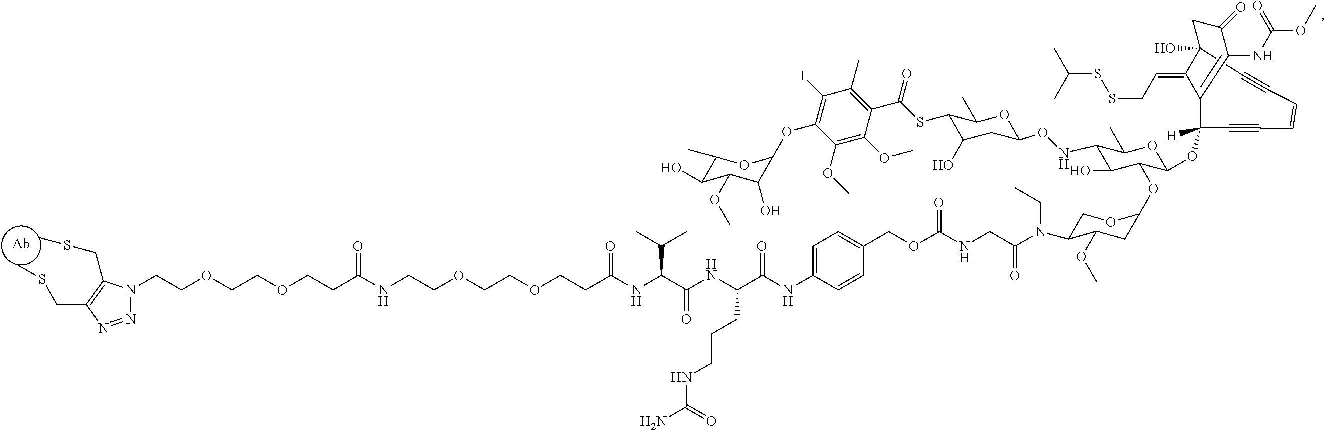
or a pharmaceutically acceptable salt thereof.

5. The ADC of claim 1, wherein the structure of Formula II has a structure selected from the group consisting of: 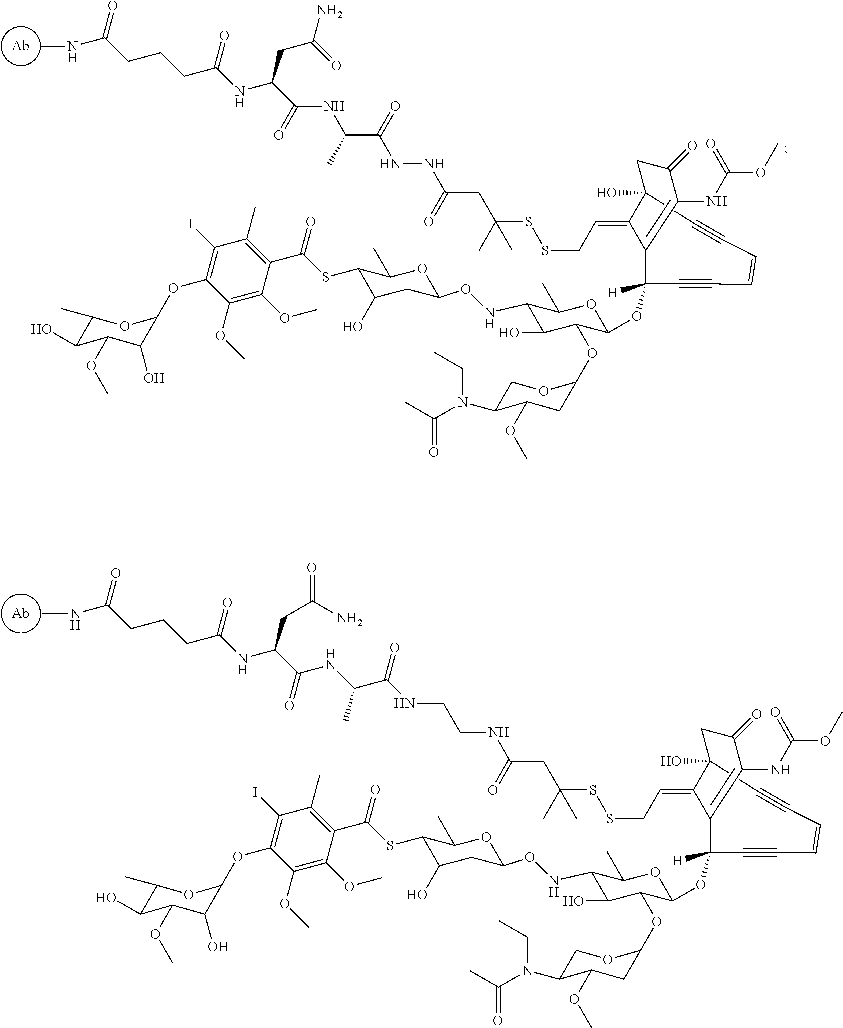 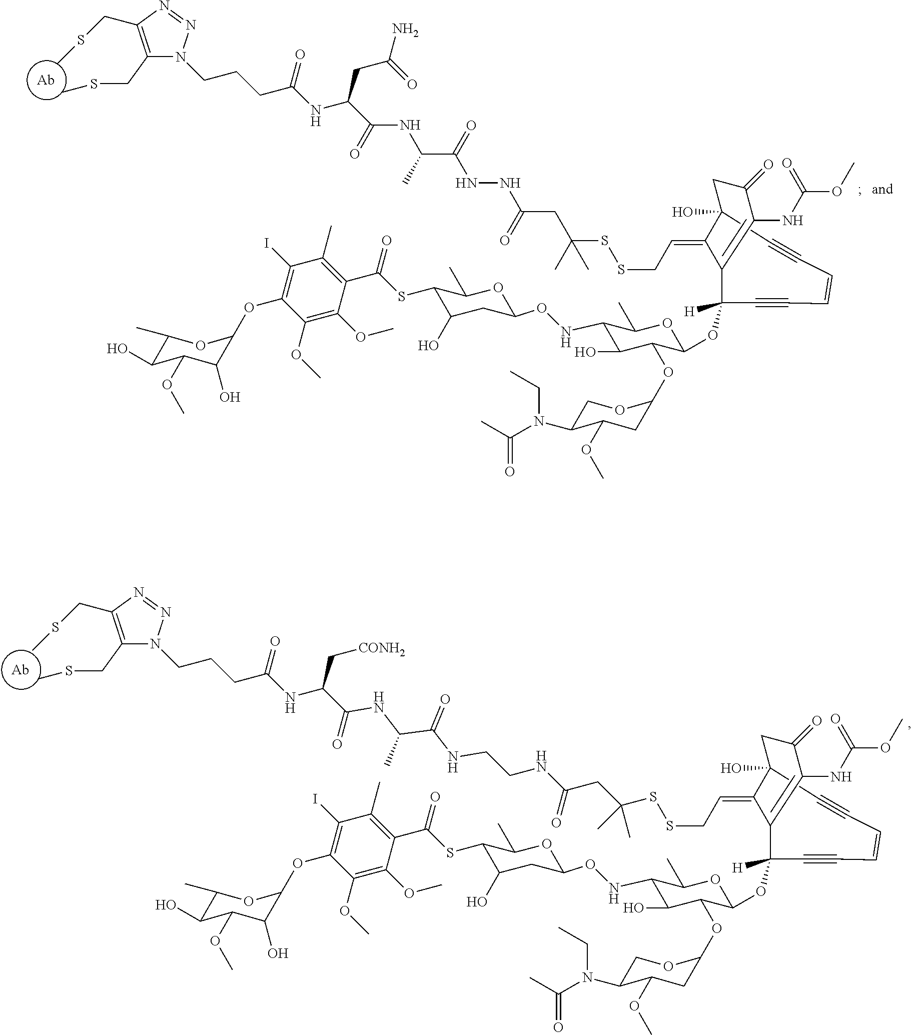
or a pharmaceutically acceptable salt thereof.

6. A compound having the structure: 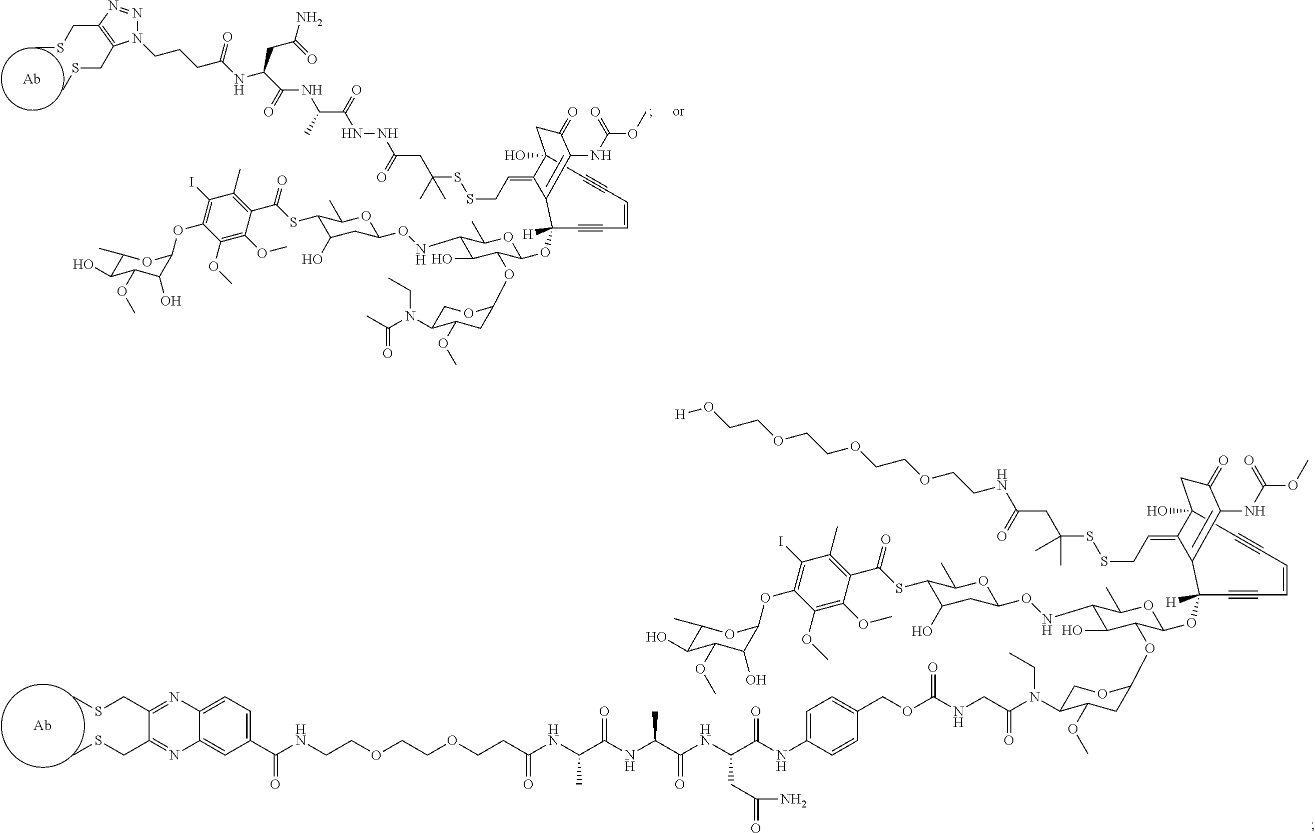
or a pharmaceutically acceptable salt thereof.

7. The compound of claim 6, wherein Ab is antibody of an IgG class that binds to a c-Met epitope with a binding affinity of at least 10⁻⁶M.
